(19) United States
(12) Patent Application Publication
(10) Pub. No.: US 2016/0339005 A1
(43) Pub. Date: Nov. 24, 2016

(54) COMPOSITIONS AND METHODS FOR TREATING OCULAR DISEASES

(71) Applicant: AKEBIA THERAPEUTICS, INC., Cambridge, MA (US)

(72) Inventors: Robert Shalwitz, Bexley, OH (US); William Daly, San Clemente, CA (US)

(73) Assignee: Akebia Therapeutics, Inc., Cambridge, MA (US)

(21) Appl. No.: 15/112,954

(22) PCT Filed: Jan. 23, 2015
(86) PCT No.: PCT/US15/12634

§ 371 Date: Jul. 20, 2016

Related U.S. Application Data
(60) Provisional application No. 61/930,811, filed on Jan. 23, 2014.

(51) Int. Cl. A61K 31/4418; A61K 31/44; A61K 31/7088; A61K 9/00; A61K 45/06

(52) U.S. Cl. CPC A61K 31/4418; A61K 9/0048; A61K 45/06; A61K 31/7088; A61K 31/44

(57) ABSTRACT

Disclosed are methods for treating diseases or conditions of the eye, especially retinopathies, ocular edema and ocular neovascularization. Non-limiting examples of these diseases or conditions include diabetic macular edema, age-related macular degeneration (wet or dry form), choroidal neovascularization, diabetic retinopathy, retinal vein occlusion (central or branch), ocular trauma, surgery induced edema, surgery induced neovascularization, cystoid macular edema, ocular ischemia, uveitis, and the like. These diseases or conditions are characterized by changes in the ocular vasculature whether progressive or non-progressive, whether a result of an acute disease or condition, or a chronic disease or condition.

The present application claims benefit of priority from U.S. provisional patent application No. 61/930,811 filed on Jan. 23, 2014, which is incorporated herein by reference in its entirety.

### 1 FIELD OF THE INVENTION

Disclosed are methods for treating diseases or conditions of the eye, especially retinopathies, ocular edema and ocular neovascularization. Non-limiting examples of these diseases or conditions include diabetic macular edema, age-related macular degeneration (wet or dry form), choroidal neovascularization, diabetic retinopathy, retinal vein occlusion (central or branch), ocular trauma, surgery induced edema, surgery induced neovascularization, cystoid macular edema, ocular ischemia, uveitis, and the like. These diseases or conditions are characterized by changes in the ocular vasculature whether progressive or non-progressive, whether a result of an acute disease or condition, or a chronic disease or condition.

### 2 BACKGROUND OF THE INVENTION

The eye comprises several structurally and functionally distinct vascular beds, which supply ocular components critical to the maintenance of vision. These include the retinal and choroidal vasculatures, which supply the inner and outer portions of the retina, respectively, and the limbal vasculature located at the periphery of the cornea. Injuries and diseases that impair the normal structure or function of these vascular beds are among the leading causes of visual impairment and blindness. For example, diabetic retinopathy is the most common disease affecting the retinal vasculature, and is the leading cause of vision loss amongst the working age population in the United States. Vascularization of the cornea secondary to injury or disease is yet another category of ocular vascular disease that can lead to severe impairment of vision.

Macular degeneration is a general medical term that applies to any of several disease syndromes which involve a gradual loss or impairment of eyesight due to cell and tissue degeneration of the yellow macular region in the center of the retina. Macular degeneration is often characterized as one of two types, non-exudative (dry form) or exudative (wet form). Although both types are bilateral and progressive, each type may reflect different pathological processes. The wet form of age-related macular degeneration (AMD) is the most common form of choroidal neovascularization and a leading cause of blindness in the elderly. AMD affects millions of Americans over the age of 60, and is the leading cause of new blindness among the elderly.

Choroidal neovascular membrane (CNVM) is a problem that is related to a wide variety of retinal diseases, but is most commonly linked to age-related macular degeneration. With CNVM, abnormal blood vessels stemming from the choroid (the blood vessel-rich tissue layer just beneath the retina) grow up through the retinal layers. These new vessels are very fragile and break easily, causing blood and fluid to pool within the layers of the retina.

Diabetes (diabetes mellitus) is a metabolic disease caused by the inability of the pancreas to produce insulin or to use the insulin that is produced. The most common types of diabetes are type 1 diabetes (often referred to as Juvenile Onset Diabetes Mellitus) and type 2 diabetes (often referred to as Adult Onset Diabetes Mellitus). Type 1 diabetes results from the body's failure to produce insulin due to loss of insulin producing cells, and presently requires the person to inject insulin. Type 2 diabetes generally results from insulin resistance, a condition in which cells fail to use insulin properly. Type 2 diabetes has a component of insulin deficiency as well. Diabetes is directly responsible for a large number of disease conditions, including conditions or diseases of the eye including diabetic retinopathy (DR) and diabetic macular edema (DME), which are leading causes of vision loss and blindness in most developed countries. The increasing number of individuals with diabetes worldwide suggests that DR and DME will continue to be major contributors to vision loss and associated functional impairment for years to come.

Diabetic retinopathy is a complication of diabetes that results from damage to the blood vessels of the light-sensitive tissue at the back of the eye (retina). At first, diabetic retinopathy may cause no symptoms or only mild vision problems. Eventually, however, diabetic retinopathy can result in blindness. Diabetic retinopathy can develop in anyone who has type 1 diabetes or type 2 diabetes.

At its earliest stage, in non-proliferative retinopathy, microaneurysms occur in the retina's tiny blood vessels. As the disease progresses, more of these blood vessels become damaged or blocked and these areas of the retina send signals into the regional tissue to grow new blood vessels for nourishment. This stage is called proliferative retinopathy. The new blood vessels grow along the retina and along the surface of the clear, vitreous gel that fills the inside of the eye. By themselves, these blood vessels do not cause symptoms or vision loss. However, they have thin, fragile walls, and without timely treatment, these new blood vessels can leak blood (whole blood or some constituents thereof) which can result in severe vision loss and even blindness. Also, fluid can leak into the center of the macula, the part of the eye where sharp, straight-ahead vision occurs. The fluid and the associated protein begin to deposit on or under the macula, swell, and the patient's central vision becomes distorted. This condition is called macular edema. It can occur at any stage of diabetic retinopathy, although it is more likely to occur as the disease progresses. About half of the people with proliferative retinopathy also have macular edema.

Uveitis is a condition in which the uvea becomes inflamed. The eye is shaped much like a tennis ball, hollow on the inside with three different layers of tissue surrounding a central cavity. The outermost is the sclera (white coat of the eye) and the innermost is the retina. The middle layer between the sclera and the retina is called the uvea. The uvea contains many of the blood vessels that nourish the eye. Complications of uveitis include glaucoma, cataracts or new blood vessel formation (neovascularization).

The currently available interventions for exudative (wet form) macular degeneration, diabetic retinopathy, diabetic macular edema, choroidal neovascular membrane and complications from uveitis or trauma, include laser photocoagulation therapy, low dose radiation (teletherapy) and surgical removal of neovascular membranes (vitrectomy). Laser therapy has had limited success, and selected choroidal neovascular membranes which initially respond to laser therapy have high disease recurrence rates. There is also a potential loss of vision resulting from laser therapy. Low dose radiation has been applied ineffectively to induce regression of choroidal neovascularization. Recently, ranibizumab and pegaptinib which are vascular endothelial growth factor (VEGF) antagonists, have been approved for use in age-related macular degeneration.

Retinal vein occlusion (RVO) is the most common retinal vascular disease after diabetic retinopathy. Depending on the area of retinal venous drainage effectively occluded, it is broadly classified as either central retinal vein occlusion (CRVO), hemispheric retinal vein occlusion (HRVO), or branch retinal vein occlusion (BRVO). It has been observed that each of these has two subtypes. Presentation of RVO in general is with variable painless visual loss with any combination of fundal findings consisting of retinal vascular tortuosity, retinal hemorrhages (blot and flame shaped), cotton wool spots, optic disc swelling and macular edema. In a CRVO, retinal hemorrhages will be found in all four quadrants of the fundus, whilst these are restricted to either the superior or inferior fundal hemisphere in a HRVO. In a BRVO, hemorrhages are largely localized to the area drained by the occluded branch retinal vein. Vision loss occurs secondary to macular edema or ischemia.

Hypoxia-inducible factor (HIF) is a transcription factor that is a key regulator of responses to hypoxia. In response to hypoxic conditions, i.e., reduced oxygen levels in the cellular environment, HIF upregulates transcription of several target genes, including vascular endothelial growth factor (VEGF). HIF is a heteroduplex comprising an α and β subunit. While the beta subunit is normally present in excess and is not dependent on oxygen tension, the HIFα subunit is only detectable in cells under hypoxic conditions. In this regard, the accumulation of HIFα is regulated primarily by hydroxylation at two proline residues by a family of prolyl hydroxylases known as HIF prolyl hydroxylases, wherein hydroxylation of one or both of the proline residues leads to the rapid degradation of HIFα. Accordingly, inhibition of HIF prolyl hydroxylase results in stabilization and accumulation of HIFα (i.e., the degradation of HIF-α is reduced), thereby leading to an increase in the amount of HIFα available for formation of the HIF heterodimer and upregulation of target genes, such as VEGF. Conversely, activation of HIF prolyl hydroxylase results in destabilization of HIFα (i.e., the degradation of HIF-α is increased), thereby leading to a decrease in the amount of HIFα available for formation of the HIF heterodimer and downregulation of target genes, such as VEGF.

A new class of prolyl hydroxylase modulators and their use to treat or prevent diseases ameliorated by modulation of hypoxia-inducible factor (HIF) prolyl hydroxylase are described in U.S. Pat. No. 7,811,595, which is incorporated herein by reference in its entirety. The synthesis of such prolyl hydroxylase inhibitors is described in U.S. Patent Publication No. 2012/0309977, which is incorporated herein by reference in its entirety.

### 3 SUMMARY OF THE INVENTION

Provided herein are methods for treating and/or preventing a disease or condition of the eye, such as those provided herein, comprising administering to a patient having a disease or condition of the eye a compound having a structure of Formula (I):

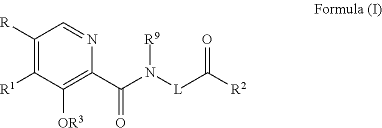

or a pharmaceutically acceptable salt, solvate or hydrate thereof, wherein

R and R¹ are each independently:
(i) hydrogen
(ii) substituted or unsubstituted phenyl; or
(iii) substituted or unsubstituted heteroaryl;
said substitution selected from:
   (i) C₁-C₄ alkyl;
   (ii) C₃-C₄ cycloalkyl;
   (iii) C₁-C₄ alkoxy;
   (iv) C₃-C₄ cycloalkoxy;
   (v) C₁-C₄ haloalkyl;
   (vi) C₃-C₄ halocycloalkyl;
   (vii) halogen;
   (viii) cyano;
   (ix) NHC(O)R⁴;
   (x) C(O)NR^{5a}R^{5b}; and
   (xi) heteroaryl; or
   (xii) two substituents are taken together to form a fused ring having from 5 to 7 atoms;

R⁴ is a C₁-C₄ alkyl or C₃-C₄ cycloalkyl;

R^{5a} and R^{5b} are each independently selected from:
(i) hydrogen;
(ii) C₁-C₄ alkyl;
(iii) C₃-C₄ cycloalkyl; or
(iv) R^{5a} and R^{5b} are taken together to form a ring having from 3 to 7 atoms;

R² is selected from:
(i) OR⁶
(ii) NR^{7a}R^{7b}; and

R⁶ is selected from hydrogen and C₁-C₄ alkyl or C₃-C₄ cycloalkyl;

R^{7a} and R^{7b} are each independently selected from:
(i) hydrogen;
(ii) C₁-C₄ alkyl or C₃-C₄ cycloalkyl; or
(iii) R^{7a} and R^{7b} are taken together to form a ring having from 3 to 7 atoms;

R³ is selected from hydrogen, methyl, and ethyl;

L is a linking unit having a structure —[C(R^{8a}R^{8b})]ₙ—

R^{8a} and R^{8b} are each independently selected from hydrogen, methyl and ethyl;

n is an integer from 1 to 3; and

R⁹ is selected from hydrogen and methyl.

Provided herein are methods for treating a disease or condition of the eye, such as those provided herein, comprising administering to a patient having a disease or condition of the eye a compound having a structure of Formula (VI):

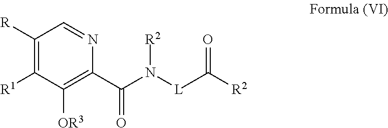

or a pharmaceutically acceptable salt, solvate or hydrate thereof, wherein
R and R¹ are each independently:
i) hydrogen
ii) substituted or unsubstituted phenyl; or
iii) substituted or unsubstituted heteroaryl;
said substitution selected from:
i) C₁-C₄ linear, branched, or cyclic alkyl;
ii) C₁-C₄ linear, branched, or cyclic alkoxy;
iii) C₁-C₄ linear, branched, or cyclic haloalkyl;
iv) halogen;
v) cyano;

### vi) NHC(O)R⁴;

vii) C(O)NR^{5a}R^{5b}; and
viii) heteroaryl; or
ix) two substituents are taken together to form a fused ring having from 5 to 7 atoms;
R⁴ is hydrogen or a C₁-C₄ linear, branched, or cyclic alkyl;
R^{5a} and R^{5b} are each independently selected from:
i) hydrogen; and
ii) C₁-C₄ linear, branched, or cyclic alkyl; or
iii) R^{5′} and R^{5b} are taken together to form a ring having from 3 to 7 atoms;
R² is selected from:

### i) OR⁶

ii) NR^{7a}R^{7b}; and
R⁶ is selected from hydrogen and C₁-C₄ linear, branched, or cyclic alkyl;
R^{7a} and R^{7b} are each independently selected from:
i) hydrogen; and
ii) C₁-C₄ linear, branched, or cyclic alkyl; or
iii) R^{7a} and R^{7b} are taken together to form a ring having from 3 to 7 atoms;
R³ is selected from hydrogen, methyl, and ethyl;
L is a linking unit having a structure

—[C(R^{8a}R^{8b})]ₙ—

R^{8a} and R^{8b} are each independently selected from hydrogen and methyl;
n is 1 or 2; and
R⁹ is selected from hydrogen and methyl;
provided that one of R and R¹ is not hydrogen.

In certain embodiments, the disease or condition of the eye is characterized by changes in the ocular vasculature.

In certain embodiments, the disease or condition of the eye is selected from retinopathy, ocular edema and ocular neovascularization.

In certain embodiments, the disease or condition of the eye is selected from diabetic macular edema, age-related macular degeneration, choroidal neovascularization, diabetic retinopathy, ocular ischemia, uveitis, retinal vein occlusion, ocular trauma, surgery induced edema, surgery-induced neovascularization, cystoid macular edema, ocular ischemia, and uveitis.

In certain embodiments, the disease or condition of the eye is age-related macular degeneration. In certain embodiments, the age-related macular edema is selected from wet age-related macular degeneration and dry age-related macular degeneration.

In certain embodiments, the patient having age-related macular degeneration is at least about 50 years old, at least about 55 years old, at least about 60 years old, at least about 65 years old or at least about 70 years old.

In certain embodiments, the disease or condition of the eye is characterized by unstable ocular vasculature of a diabetic patient.

In certain embodiments, the disease or condition of the eye is diabetic macular edema.

In certain embodiments, the disease or condition of the eye is diabetic retinopathy. In certain embodiments, the diabetic retinopathy is proliferative. In certain embodiments, the diabetic retinopathy is non-proliferative.

In certain embodiments, the disease or condition of the eye is retinal vein occlusion.

In certain embodiments, the disease or condition of the eye can be a condition selected from the group consisting of retinopathy, diabetic retinopathy, radiation retinopathy, macular degeneration, age-related macular degeneration, early stage age-related macular degeneration, intermediate stage age-related macular degeneration, advanced stage age-related macular degeneration, Wet (exudative) age-related macular degeneration, specific genotypes associated with macular degeneration, cancer, solid tumors, blood borne tumors, choroidal melanoma, sickle cell retinopathy, neovascularization, ocular neovascularization, subretinal neovascularization, vein occlusion, retinopathy of prematurity, chronic uveitis/vitritis, ocular trauma, ocular ischemia, retinal ischemia, Best's disease, chronic retinal detachment, diseases associated with rubeosis, Eales' disease, proliferative vitreoretinopathy, familial exudative vitreoretinopathy, Stargardt's disease, presumed ocular histoplasmosis, hyperviscosity syndromes, myopia, post-laser complications, retinopathy of prematurity, infections causing a retinitis or choroiditis, optic pits, pars planitis, toxoplasmosis, choroidal neovascularization, Type 1 choroidal neovascularization, Type 2 choroidal neovascularization, Type 3 choroidal neovascularization, macular edema, cystoid macular edema, diabetic macular edema, ocular edema, glaucoma, neovascular glaucoma, surgery-induced edema, surgery-induced neovascularization, retinoschisis, retinal capillary occlusions, retinal angiomatous proliferation, vitreous hemorrhage, retinal neovascularization, polypoidal choroidal vasculopathy, juxtafoveal polypoidal choroidal vasculopathy, subfovial polypoidal choroidal vasculopathy, vitreomacular adhesion, geographic atrophy, retinal hypoxia, pathological myopia, dysregulated para-inflammation, chronic inflammation, chronic wound healing environment in the aging eye, carotid vacernous fistula, idiopathic occlusive arteriolitis, birdshot retinochoroidopathy, retinal vasculitis, incontinentia pigmenti, retinitis pigmentosa, tachyphylaxis, and limbal stem cell deficiency.

In certain embodiments, the disease or condition of the eye can be a condition selected from the group consisting of radiation retinopathy, age-related macular degeneration, early stage age-related macular degeneration, intermediate stage age-related macular degeneration, advanced stage age-related macular degeneration, Wet (exudative) age-related macular degeneration, specific genotypes associated with macular degeneration, cancer, choroidal melanoma, sickle cell retinopathy, subretinal neovascularization, choroidal neovascularization, Type 1 choroidal neovascularization, Type 2 choroidal neovascularization, Type 3 choroidal neovascularization, macular edema, cystoid macular edema, diabetic macular edema, ocular edema, glaucoma, neovascular glaucoma, surgery-induced edema, surgery-induced neovascularization, retinoschisis, retinal capillary occlusions, retinal angiomatous proliferation, vitreous hemorrhage, retinal neovascularization, polypoidal choroidal vasculopathy, juxtafoveal polypoidal choroidal vasculopathy, subfovial polypoidal choroidal vasculopathy, vitreomacular adhesion, geographic atrophy, retinal hypoxia, pathological myopia, dysregulated para-inflammation, chronic inflammation, chronic wound healing environment in the aging eye, carotid vacernous fistula, idiopathic occlusive arteriolitis, birdshot retinochoroidopathy, retinal vasculitis, incontinentia pigmenti, retinitis pigmentosa, tachyphylaxis, and limbal stem cell deficiency.

In certain embodiments, the compound of Formula (I) has a structure:

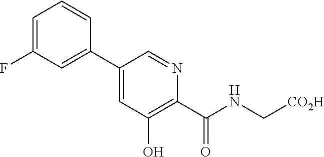 [SMILES: O=C(O)CNC(=O)c1ncc(-c2cccc(F)c2)cc1O]

or a pharmaceutically acceptable salt, solvate or hydrate thereof

In certain embodiments, the compound of Formula (I) is administered intravitreally or topically.

In certain embodiments, the compound of Formula (I) is administered in combination with another medicament.

In certain embodiments, the other medicament is selected from a prostaglandin analog, beta-adrenergic receptor antagonist, alpha-2-adrenergic agonist, carbonic anhydrase inhibitor, miotic agent, monoclonal antibody, corticosteroid, glucocorticoid, kinase inhibitor, cycloplegic and antimetabolite, or a combination thereof

In certain embodiments, the other medicament is an anti-angiogenic medicament. In certain embodiments, the anti-angiogenic medicament selected from bevacizumab, aflibercept, ranibizumab, or pegaptanib sodium.

In certain embodiments, the other medicament is laser therapy.

In certain embodiments, the other medicament is an anti-platelet-derived growth factor (anti-PDGF) agent. In certain embodiments, the anti-platelet-derived growth factor agent is pegpleranib sodium. In certain embodiments, the anti-platelet-derived growth factor agent is Fovista®.

In certain embodiments, the other medicament is an anti-vascular endothelial growth factor (anti-VEGF) agent. In certain embodiments, the anti-vascular endothelial growth factor agent is Lucentis®, Avastin® Eylea®, or Macugen®.

### 4 BRIEF DESCRIPTION OF THE FIGURES

FIG. 1 depicts fluorescein angiography images of an animal treated with vehicle control. Images were obtained prior to dosing at Day 3 (top), and then at Day 9 (center), and Day 14 (bottom). The arrow in the Day 3 (top) image indicates excessive leakage and high fluorescence. Continued fluorescence leakage was observed on Day 9 and Day 14. The arrow in the Day 14 (bottom) image indicates continued fluorescein leakage.

FIG. 2 depicts fluorescein angiography images of an animal treated with a low dose (5 μL at 3.7 mg/mL) of Compound 7. Images were obtained prior to dosing at Day 3 (top), and then at Day 9 (center), and Day 14 (bottom). The arrow in the Day 3 (top) image indicates excessive leakage and high fluorescence. Lower fluorescence leakage was observed on Day 9, and minimal fluorescence leakage was observed on Day 14. The arrow in the Day 14 (bottom) image indicates minimal fluorescein leakage.

FIG. 3 depicts fluorescein angiography images of an animal treated with a high dose (5 μL at 7.8 mg/mL) of Compound 7. Images were obtained prior to dosing at Day 3 (top), and then at Day 9 (center), and Day 14 (bottom). The arrow in the Day 3 (top) image indicates excessive leakage and high fluorescence. Lower fluorescence leakage was observed on Day 9, and no fluorescence leakage was observed on Day 14. The arrow in the Day 14 (bottom) image indicates an area of no fluorescein leakage.

FIG. 4 depicts a reduction of vascular leakage in the ocular vasculature in animals treated with a low dose (5 μL at 3.7 mg/mL) of Compound 7 (medium grey bars, center) and animals treated with a high dose (5 μL at 7.8 mg/mL) of Compound 7 (dark grey bars, right) compared with animals treated with vehicle control (light grey bars, left). The data indicate Mean (±SEM) of lesion areas OD (right eye) over time.

FIG. 5 depicts a reduction of vascular leakage in the ocular vasculature in animals treated with a low dose (5 μL at 3.7 mg/mL) of Compound 7 (medium grey bars, center) and animals treated with a high dose of Compound 7 (dark grey bars, right) compared with animals treated with vehicle control (light grey bars, left). The data indicate Mean (±SEM) of lesion areas OS (left eye) over time.

FIG. 6 depicts a reduction of vascular leakage in the ocular vasculature in animals treated with a low dose (5 μL at 3.7 mg/mL) of Compound 7 (medium grey bars, center) and animals treated with a high dose (5 μL at 7.8 mg/mL) of Compound 7 (dark grey bars, right) compared with animals treated with vehicle control (light grey bars, left). The data indicate Mean (±SEM) of lesion areas OU (both eyes) over time.

### 5 DETAILED DESCRIPTION

### 5.1 Definitions

As used herein, the term “dose(s)” means a quantity of the compound or a pharmaceutically acceptable salt, solvate, or hydrate thereof to be administered at one time. A dose may comprise a single unit dosage form, or alternatively may comprise more than a single unit dosage form (e.g., a single dose may comprise two tablets), or even less than a single unit dosage form (e.g., a single dose may comprise half of a tablet).

As used herein, the term “daily dose” means a quantity of the compound, or a pharmaceutically acceptable salt, solvate, or hydrate thereof that is administered in a 24 hour period. Accordingly, a daily dose may be administered all at once (i.e., once daily dosing) or alternatively the daily dosing may be divided such that administration of the compound is twice daily, three times daily, or even four times daily.

As used herein, the term “patient” or “subject” means a human.

As used herein, an “effective amount” refers to that amount of a compound disclosed herein that is sufficient to provide a therapeutic benefit in the treatment of the disease or to delay or minimize symptoms associated with the disease. In certain embodiments the disease is a disease or condition of the eye.

As used herein, the terms “prevent”, “preventing” and “prevention” are art-recognized, and when used in relation to a condition, such as a disease or condition of the eye, or any other medical condition, such as those described herein, is well understood in the art, and includes administration of a compound which reduces the frequency of, or delays the onset of, symptoms of a medical condition in a subject relative to a subject which does not receive the composition.

As used herein, the terms “treat”, “treating” and “treatment” refer to the reversing, reducing, or arresting the symptoms, clinical signs, and underlying pathology of a condition in manner to improve or stabilize a subject's condition. The terms “treat” and “treatment” also refer to the eradication or amelioration of the disease or symptoms associated with the disease. In certain embodiments, such terms refer to minimizing the spread or worsening of the disease resulting from the administration of a compound as disclosed herein to a patient with such a disease. In certain embodiments the disease is a disease or condition of the eye.

As used herein, the term “pharmaceutically acceptable salt” refers to a salt prepared from pharmaceutically acceptable non-toxic acids or bases including inorganic acids and bases and organic acids and bases. Suitable pharmaceutically acceptable base addition salts include, but are not limited to, sodium, lithium, potassium, calcium, magnesium, zinc, bismuth, ammonium (including alkyl substituted ammonium), amino acids (e.g., lysine, ornithine, arginine, or glutamine), tromethamine, and meglumine. Suitable non-toxic acids include, but are not limited to, inorganic and organic acids such as acetic, alginic, anthranilic, benzenesulfonic, benzoic, camphorsulfonic, citric, ethanesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Other examples of salts are well known in the art, see, e.g., Remington's Pharmaceutical Sciences, 22nd ed., Pharmaceutical Press, (2012).

As used herein, the term “hydrate” means a compound as disclosed herein, that further includes a stoichiometric or non-stoichiometric amount of water bound by non-covalent intermolecular forces.

As used herein, the term “solvate” means a compound as disclosed herein, that further includes a stoichiometric or non-stoichiometric amount of a solvent, other than water, bound by non-covalent intermolecular forces.

As used herein, the term “HIF prolyl hydroxylase” is art-recognized and may be abbreviated as “PHD”. HIF prolyl hydroxylase is also known as “prolyl hydroxylase domain-containing protein” which may be abbreviated as “PHD”. In this regard, there are three different PHD isoforms, PHD1, PHD2, and PHD3, also referred to as EGLN2, EGLN1, and EGLN3, or HPH3, HPH2, and HPH1, respectively.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 15%, 10%, 9%, 8%, 7%, 6%, 5%, 4%, 3%, 2%, 1%, 0.5%, or 0.05% of a given value or range.

As used herein, the term “Compound 7” refers to a compound, {[5-(3-fluorophenyl)-3-hydroxypyridine-2-carbonyl]amino} acetic acid, having the structure

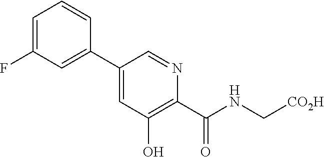 [SMILES: O=C(O)CNC(=O)c1ncc(-c2cccc(F)c2)cc1O]

In certain embodiments, the compound may be {[5-(3-fluorophenyl)-3-hydroxypyridine-2-carbonyl]amino} acetic acid, while in certain alternative embodiments, the compound may be a pharmaceutically acceptable salt of {[5-(3-fluorophenyl)-3-hydroxypyridine-2-carbonyl]amino} acetic acid. In certain alternative embodiments, the compound may be a solvate of {[5-(3-fluorophenyl)-3-hydroxypyridine-2-carbonyl]amino}acetic acid. In certain alternative embodiments, the compound may be a hydrate of {[5-(3-fluorophenyl)-3-hydroxypyridine-2-carbonyl]amino}acetic acid. In certain preferred embodiments, the invention relates to the compound in its parent form (i.e., not a salt, solvate, or hydrate). In certain alternative preferred embodiments, the invention relates to the compound or a pharmaceutically acceptable salt thereof

As used herein, the term “alkyl” refers to a saturated or partially saturated straight chain or branched non-cyclic hydrocarbon having from 1 to 4 carbon atoms.

As used herein, the term “cyclic alkyl” refers to a saturated or partially saturated cyclic alkyl group.

As used herein, the term “alkoxy” refers to an —O (alkyl), wherein alkyl is defined above.

As used herein, the term “haloalkyl” refers to an alkyl as defined above, substituted with one or more of chloro, iodo, bromo, or fluoro.

As used herein, the term “heteroaryl” refers to an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.2 Compounds

Compounds that can be used with the compositions and formulations provided herein are modulators of a HIF prolyl hydroxylase. In more specific embodiments, a compound for use with the methods provided herein is a modulator of a HIF-1-alpha prolyl hydroxylase. In other, more specific embodiments, a compound for use with the methods provided herein is a modulator of a HIF-2-alpha prolyl hydroxylase. In certain, even more specific embodiments, a compound for use with the methods provided herein is a modulator of a HIF-2-alpha prolyl hydroxylase that is more active against HIF-2-alpha prolyl hydroxylase than HIF-1-alpha prolyl hydroxylase by at least 10%, 20%, 25%, 30%, 40%, 50%, 60%, 70%, 75%, 80%, 90%, 100%, 125%, 150%, 175%, 200%, 250%, 500%, 750%, or at least 1000%. Thus, in certain embodiments, a compound provided herein for use with the methods provided herein preferentially stabilizes HIF-2-alpha over HIF-1-alpha. To determine preferential stabilization of HIF-2-alpha over HIF-1-alpha, the concentrations of HIF-1-alpha and HIF-2-alpha in a subject with and without test compound can be determined using a HIF-1-alpha and a HIF-2-alpha ELISA kit. Care should be taken that the primary antibodies in the respective kits are not cross-reactive with the other HIF (i.e., the primary antibody against HIF-1-alpha reacts immunospecifically with HIF-1-alpha and does not cross-react with HIF-2-alpha; the primary antibody against HIF-2-alpha reacts immunospecifically with HIF-2-alpha and does not cross-react with HIF-1-alpha).

In certain embodiments, a compound of the invention which is a HIF prolyl hydroxylase inhibitor or a HIF-alpha stabilizer is a heterocyclic carboxamide. In certain such embodiments, the heterocyclic carboxamide is selected from a pyridyl carboxamide, a quinoline carboxamide, and an isoquinoline carboxamide.

In certain embodiments, the HIF prolyl hydroxylase inhibitor or HIF-alpha stabilizer has a structure of Formula (I):

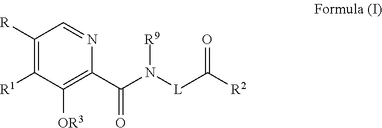

or a pharmaceutically acceptable salt, solvate or hydrate thereof, wherein

R and R¹ are each independently:
(i) hydrogen
(ii) substituted or unsubstituted phenyl; or
(iii) substituted or unsubstituted heteroaryl;
said substitution selected from:
   (i) C₁-C₄ alkyl;
   (ii) C₃-C₄ cycloalkyl;
   (iii) C₁-C₄ alkoxy;
   (iv) C₃-C₄ cycloalkoxy;
   (v) C₁-C₄ haloalkyl;
   (vi) C₃-C₄ halocycloalkyl;
   (vii) halogen;
   (viii) cyano;
   (ix) NHC(O)R⁴;
   (x) C(O)NR^{5a}R^{5b}; and
   (xi) heteroaryl; or
   (xii) two substituents are taken together to form a fused ring having from 5 to 7 atoms;

R⁴ is a C₁-C₄ alkyl or C₃-C₄ cycloalkyl;

R^{5a} and R^{5b} are each independently selected from:
(i) hydrogen;
(ii) C₁-C₄ alkyl;
(iii) C₃-C₄ cycloalkyl; or
(iv) R^{5a} and R^{5b} are taken together to form a ring having from 3 to 7 atoms;

R² is selected from:
(i) OR⁶
(ii) NR^{7a}R^{7b}; and

R⁶ is selected from hydrogen and C₁-C₄ alkyl or C₃-C₄ cycloalkyl;

R^{7a} and R^{7b} are each independently selected from:
(i) hydrogen;
(ii) C₁-C₄ alkyl or C₃-C₄ cycloalkyl; or
(iii) R^{7a} and R^{7b} are taken together to form a ring having from 3 to 7 atoms;

R³ is selected from hydrogen, methyl, and ethyl;

L is a linking unit having a structure —[C(R^{8a}R^{8b})]ₙ—

R^{8a} and R^{8b} are each independently selected from hydrogen, methyl and ethyl;

n is an integer from 1 to 3; and

R⁹ is selected from hydrogen and methyl.

In certain, more specific embodiments, in Formula (I) R and R¹ are not both hydrogen.

In certain embodiments, the HIF prolyl hydroxylase inhibitor or HIF-alpha stabilizer has a structure of Formula (II):

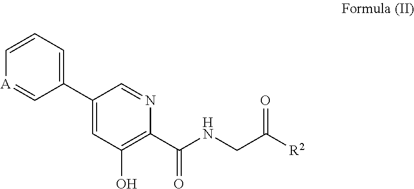

or a pharmaceutically acceptable salt, solvate or hydrate thereof, wherein
A is selected from the group consisting of CR′, N, N⁺—O⁻ and N⁺(C₁-C₆ alkyl);
R′ is selected from the group consisting of H, C₁-C₆ alkyl, C₃-C₆ cycloalkyl, C₂-C₆ alkenyl, C₃-C₆ cycloalkenyl, C₂-C₆ alkynyl, C₄-C₇ heterocycloalkyl, C₆-C₁₀ aryl, C₅-C₁₀ heteroaryl, NH₂, NHR″, N(R″)₂, NHC(O)R″, NR″C(O)R″, F, Cl, Br, I, OH, OR″, SH, SR″, S(O)R″, S(O)₂R″, S(O)NHR″, S(O)₂NHR″, S(O)NR″₂, S(O)₂NR″₂, C(O)R″, CO₂H, CO₂R″, C(O)NH₂, C(O)NHR″, C(O)NR″₂, CN, CH₂CN, CF₃, CHF₂, CH₂F, NH(CN), N(CN)₂, CH(CN)₂, C(CN)₃; and
R″ is independently selected from the group consisting of C₁-C₆ alkyl, C₃-C₆ cycloalkyl, C₄-C₇ heterocycloalkyl, C₆-C₁₀ aryl and C₅-C₁₀ heteroaryl; and wherein C₁-C₆ alkyl, C₃-C₆ cycloalkyl, or C₄-C₇ heterocycloalkyl are optionally substituted with oxo, NH₂, NHR″, N(R″)₂, F, Cl, Br, I, OH, OR″, SH, SR″, S(O)R″, S(O)₂R″, S(O)NHR″, S(O)₂NHR″, S(O)NR″₂, S(O)₂NR″₂, C(O)R″, CO₂H, CO₂R″, C(O)NH₂, C(O)NHR″, C(O)NR″₂, CN, CH₂CN, CF₃, CHF₂, CH₂F, NH(CN), N(CN)₂, CH(CN)₂, C(CN)₃; and wherein C₆-C₁₀ aryl or C₅-C₁₀ heteroaryl are optionally substituted with C₁-C₆ alkyl, C₃-C₆ cycloalkyl, C₂-C₆ alkenyl, C₃-C₆ cycloalkenyl, C₂-C₆ alkynyl, C₄-C₇ heterocycloalkyl, C₆ aryl, C₅-C₆ heteroaryl, NH₂, NHR″, N(R″)₂, NHC(O)R″, NR″C(O)R″, F, Cl, Br, I, OH, OR″, SH, SR″, S(O)R″, S(O)₂R″, S(O)NHR″, S(O)₂NHR″, S(O)NR″₂, S(O)₂NR″₂, C(O)R″, CO₂H, CO₂R″, C(O)NH₂, C(O)NHR″, C(O)NR″₂, CN, CH₂CN, CF₃, CHF₂, CH₂F, NH(CN), N(CN)₂, CH(CN)₂, or C(CN)₃; and wherein two R″ groups on a nitrogen can be taken together to form a ring having from 2 to 7 carbon atoms and from 1 to 3 heteroatoms chosen from nitrogen, oxygen and sulfur including the nitrogen atom to which the two R″ groups are bonded;
R² is selected from:
   (i) OR⁶;
   (ii) NR^{7a}R^{7b}; and
R⁶ is selected from hydrogen and C₁-C₁ alkyl or C₃-C₄ cycloalkyl;
R^{7a} and R^{7b} are each independently selected from:
   (i) hydrogen;
   (ii) C₁-C₁ alkyl or C₃-C₄ cycloalkyl; or
   (iii) R^{7a} and R^{7b} are taken together to form a ring having from 3 to 7 atoms

In certain embodiments, the HIF stabilizer is a compound having a structure of Formula (III)

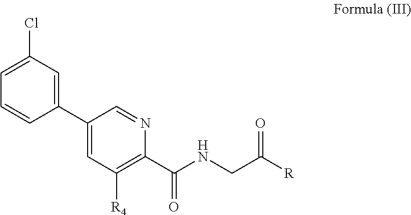

or a pharmaceutically acceptable salt, solvate or hydrate thereof, wherein

R is chosen from
(i) —OR % or
(ii) —NR²R³; or
(iii) —OM¹;

R¹ is:
(i) hydrogen; or
(ii) C₁-C₆ alkyl or C₃-C₆ cycloalkyl;

R² and R³ are each independently selected from:
(i) hydrogen;
(ii) C₁-C₁ alkyl or C₃-C₄ cycloalkyl; or
(iii) R² and R³ can be taken together to form a ring having from 2 to 7 carbon atoms and from 1 to 3 heteroatoms chosen from nitrogen, oxygen and sulfur including the nitrogen atom to which R² and R³ are bonded; and

M¹ is a cation; and

R⁴ is:
(i) —OH; or
(ii) —OM²; and

M² is a cation.

In certain embodiments, the HIF stabilizer is a compound having a structure of Formula (IV)

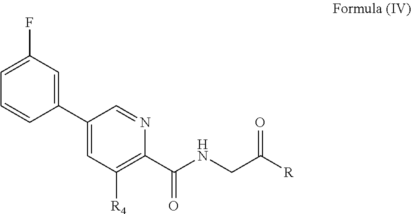

or a pharmaceutically acceptable salt, solvate or hydrate thereof, wherein

R is chosen from
(i) —OR % or
(ii) —NR²R³; or
(iii) —OM¹;

R¹ is:
(i) hydrogen; or
(ii) C₁-C₆ alkyl or C₃-C₆ cycloalkyl;

R² and R³ are each independently selected from:
(i) hydrogen;
(ii) C₁-C₁ alkyl or C₃-C₄ cycloalkyl; or
(iii) R² and R³ can be taken together to form a ring having from 2 to 7 carbon atoms and from 1 to 3 heteroatoms chosen from nitrogen, oxygen and sulfur including the nitrogen atom to which R2 and R3 are bonded; and

M¹ is a cation; and

R⁴ is:
(i) —OH; or
(ii) —OM²; and

M² is a cation.

HIF prolyl hydroxylase inhibitor compounds described herein are unsubstituted or substituted 3-hydroxy-pyridine-2-carboxamides, having the structure shown in Formula (V) below:

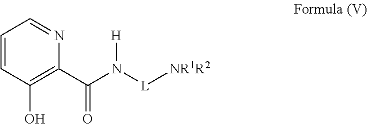

and pharmaceutically acceptable salts and tautomers thereof, wherein: L is C₁₋₆ alkyl; and wherein R¹ and R² are independently H or C₁₋₆ alkyl.

In certain embodiments, the HIF prolyl hydroxylase inhibitor or HIF-alpha stabilizer is {[5-(3-chlorophenyl)-3-hydroxypyridine-2-carbonyl]amino}acetic acid (Compound 1):

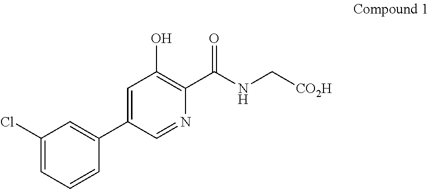 [SMILES: O=C(O)CNC(=O)c1ncc(-c2cccc(Cl)c2)cc1O]

or a pharmaceutically acceptable salt, solvate or hydrate thereof

In certain embodiments, the HIF stabilizer is Compound 2 having the structure:

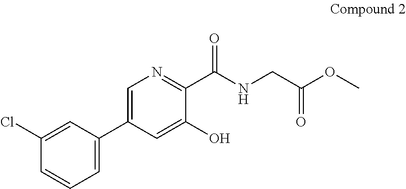 [SMILES: COC(=O)CNC(=O)c1ncc(-c2cccc(Cl)c2)cc1O]

or a pharmaceutically acceptable salt, solvate or hydrate thereof

In certain embodiments, the HIF stabilizer is Compound 3 having a structure

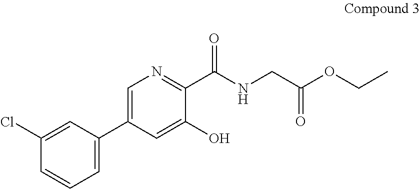 [SMILES: CCOC(=O)CNC(=O)c1ncc(-c2cccc(Cl)c2)cc1O]

or a pharmaceutically acceptable salt, solvate or hydrate thereof

In certain embodiments, the HIF stabilizer is Compound 4 having a structure

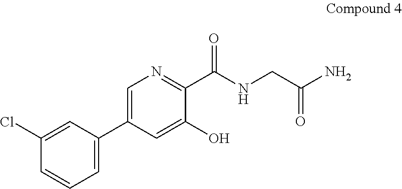 [SMILES: NC(=O)CNC(=O)c1ncc(-c2cccc(Cl)c2)cc1O]

or a pharmaceutically acceptable salt, solvate or hydrate thereof

In certain embodiments, the HIF stabilizer is Compound 5 having the structure

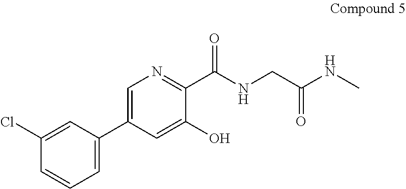 [SMILES: CNC(=O)CNC(=O)c1ncc(-c2cccc(Cl)c2)cc1O]

or a pharmaceutically acceptable salt, solvate or hydrate thereof

In certain embodiments, the HIF stabilizer is Compound 6 having the structure

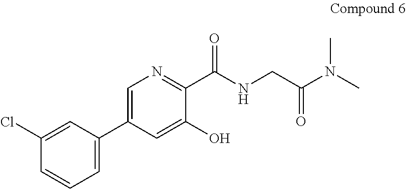 [SMILES: CN(C)C(=O)CNC(=O)c1ncc(-c2cccc(Cl)c2)cc1O]

or a pharmaceutically acceptable salt, solvate or hydrate thereof

In certain embodiments, the HIF prolyl hydroxylase inhibitor or HIF-alpha stabilizer is Compound 7 having the structure:

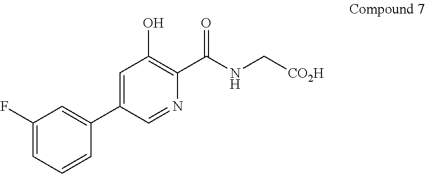 [SMILES: O=C(O)CNC(=O)c1ncc(-c2cccc(F)c2)cc1O]

or a pharmaceutically acceptable salt, solvate or hydrate thereof

In certain embodiments, the HIF stabilizer is Compound 8 having the structure:

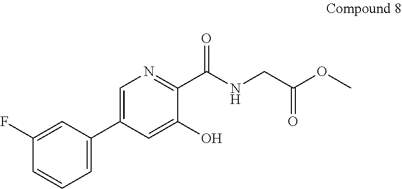 [SMILES: COC(=O)CNC(=O)c1ncc(-c2cccc(F)c2)cc1O]

or a pharmaceutically acceptable salt, solvate or hydrate thereof

In certain embodiments, the HIF stabilizer is Compound 9 having a structure

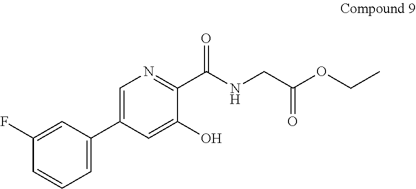 [SMILES: CCOC(=O)CNC(=O)c1ncc(-c2cccc(F)c2)cc1O]

or a pharmaceutically acceptable salt, solvate or hydrate thereof

In certain embodiments, the HIF stabilizer is Compound 10 having a structure

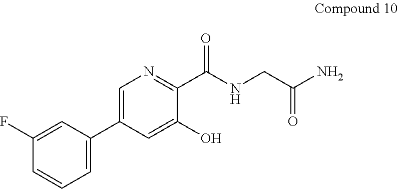 [SMILES: NC(=O)CNC(=O)c1ncc(-c2cccc(F)c2)cc1O]

or a pharmaceutically acceptable salt, solvate or hydrate thereof

In certain embodiments, the HIF stabilizer is Compound 11 having the structure

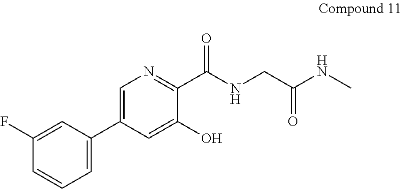 [SMILES: CNC(=O)CNC(=O)c1ncc(-c2cccc(F)c2)cc1O]

or a pharmaceutically acceptable salt, solvate or hydrate thereof

In certain embodiments, the HIF stabilizer is Compound 12 having the structure

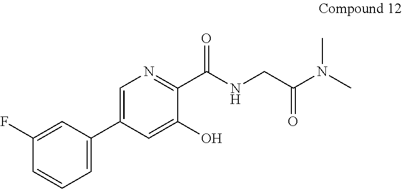 [SMILES: CN(C)C(=O)CNC(=O)c1ncc(-c2cccc(F)c2)cc1O]

or a pharmaceutically acceptable salt, solvate or hydrate thereof

In certain embodiments, the HIF stabilizer is Compound 13 having the structure

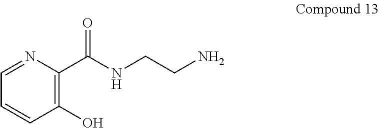 [SMILES: NCCNC(=O)c1ncccc1O]

having a name N-(2-aminoethyl)-3-hydroxy-pyridine-2-carboxamide, including pharmaceutically acceptable salts and tautomers thereof. Tautomers of Compound 13 include the following:

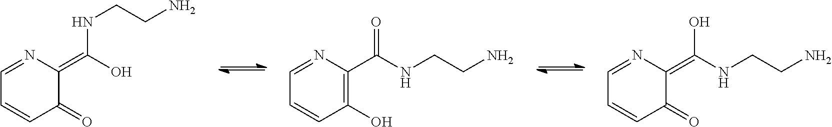 [SMILES: NCCN/C(O)=C1\N=CC=CC1=O ; NCCNC(=O)c1ncccc1O ; NCCN/C(O)=C1/N=CC=CC1=O]

In certain embodiments, a metabolite of a compound having a structure of Formula (I), Formula (II), Formula (III), Formula (IV), or of Formula (V), or a compound selected from Compound 1, Compound 2, Compound 3, Compound 4, Compound 5, Compound 6, Compound 7, Compound 8, Compound 9, Compound 10, Compound 11, Compound 12, or Compound 13 can be used with the methods provided herein. In certain more specific embodiments, such a metabolite is a phenolic glucuronide or an acyl-glucuronide.

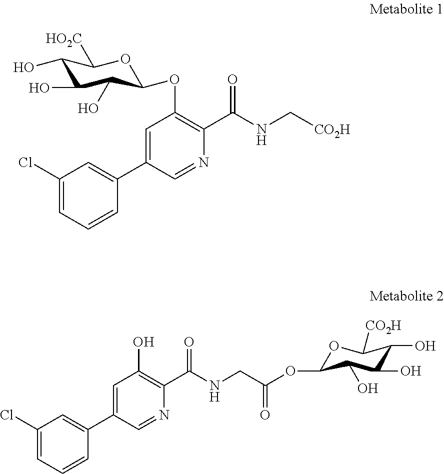

Compound 13 can be prepared using reagents and methods known in the art, including the methods provided in Chinese Patent Application Publication No. CN 85107182 A, published on Apr. 8, 1987, and German Patent Application Publication No. DE 3530046 A1, published on Mar. 13, 1986, the entire contents of each of which are incorporated herein by reference.

### 5.3 Methods of Treatment and Prevention

Neovascularization stimulated by vascular endothelial growth factor (VEGF) occurs in several important clinical contexts, including diseases or conditions characterized by changes in the ocular vasculature, including both progressive and non-progressive diseases or conditions of the eye. Thus, in certain embodiments, the invention relates to a method for treating and/or preventing a disease or condition characterized by changes in the ocular vasculature, comprising administering a compound as disclosed herein, such as Compound 7 to a patient having a disease or condition characterized by changes in the ocular vasculature.

In certain embodiments, the invention relates to a method for treating or preventing a disease or condition of the eye, comprising administering to a patient having a disease or condition of the eye, or to a patient at risk of developing a disease or condition of the eye, a pharmaceutically effective amount of a compound having a structure of Formula (I), Formula (II), Formula (III), Formula (IV), or of Formula (V), or a compound selected from Compound 1, Compound 2, Compound 3, Compound 4, Compound 5, Compound 6, Compound 7, Compound 8, Compound 9, Compound 10, Compound 11, Compound 12, and Compound 13 or a metabolite, e.g. Metabolite 1 or Metabolite 2, pharmaceutically acceptable salt, solvate, or hydrate thereof

In certain embodiments, the invention relates to a method for treating a disease or condition of the eye, comprising administering to a patient having a disease or condition of the eye, a pharmaceutically effective amount of a compound having a structure of Formula (I), Formula (II), Formula (III), Formula (IV), or of Formula (V), or a compound selected from Compound 1, Compound 2, Compound 3, Compound 4, Compound 5, Compound 6, Compound 7, Compound 8, Compound 9, Compound 10, Compound 11, Compound 12, and Compound 13 or a metabolite, e.g. Metabolite 1 or Metabolite 2, pharmaceutically acceptable salt, solvate, or hydrate thereof, wherein the condition or disease can be a condition selected from the group consisting of retinopathy, diabetic retinopathy, radiation retinopathy, macular degeneration, age-related macular degeneration early stage age-related macular degeneration, intermediate stage age-related macular degeneration, advanced stage age-related macular degeneration, Wet (exudative) age-related macular degeneration, specific genotypes associated with macular degeneration, cancer, solid tumors, blood borne tumors, choroidal melanoma, sickle cell retinopathy, neovascularization, ocular neovascularization, subretinal neovascularization, vein occlusion, retinopathy of prematurity, chronic uveitis/vitritis, ocular trauma, ocular ischemia, retinal ischemia, Best's disease, chronic retinal detachment, diseases associated with rubeosis, Eales' disease, proliferative vitreoretinopathy, familial exudative vitreoretinopathy, Stargardt's disease, presumed ocular histoplasmosis, hyperviscosity syndromes, myopia, post-laser complications, retinopathy of prematurity, infections causing a retinitis or choroiditis, optic pits, pars planitis, toxoplasmosis, choroidal neovascularization, Type 1 choroidal neovascularization, Type 2 choroidal neovascularization, Type 3 choroidal neovascularization, macular edema, cystoid macular edema, diabetic macular edema, ocular edema, glaucoma, neovascular glaucoma, surgery-induced edema, surgery-induced neovascularization, retinoschisis, retinal capillary occlusions, retinal angiomatous proliferation, vitreous hemorrhage, retinal neovascularization, polypoidal choroidal vasculopathy, juxtafoveal polypoidal choroidal vasculopathy, subfovial polypoidal choroidal vasculopathy, vitreomacular adhesion, geographic atrophy, retinal hypoxia, pathological myopia, dysregulated para-inflammation, chronic inflammation, chronic wound healing environment in the aging eye, carotid vacernous fistula, idiopathic occlusive arteriolitis, birdshot retinochoroidopathy, retinal vasculitis, incontinentia pigmenti, retinitis pigmentosa, tachyphylaxis, and limbal stem cell deficiency.

In certain embodiments, the invention relates to a method for preventing a disease or condition of the eye, comprising administering to a patient at risk of developing a disease or condition of the eye, a pharmaceutically effective amount of a compound having a structure of Formula (I), Formula (II), Formula (III), Formula (IV), or of Formula (V), or a compound selected from Compound 1, Compound 2, Compound 3, Compound 4, Compound 5, Compound 6, Compound 7, Compound 8, Compound 9, Compound 10, Compound 11, Compound 12, and Compound 13 or a metabolite, e.g. Metabolite 1 or Metabolite 2, pharmaceutically acceptable salt, solvate, or hydrate thereof, wherein the condition or disease can be a condition selected from the group consisting of retinopathy, diabetic retinopathy, radiation retinopathy, macular degeneration, age-related macular degeneration, early stage age-related macular degeneration, intermediate stage age-related macular degeneration, advanced stage age-related macular degeneration, Wet (exudative) age-related macular degeneration, specific genotypes associated with macular degeneration, cancer, solid tumors, blood borne tumors, choroidal melanoma, sickle cell retinopathy, neovascularization, ocular neovascularization, subretinal neovascularization, vein occlusion, retinopathy of prematurity, chronic uveitis/vitritis, ocular trauma, ocular ischemia, retinal ischemia, Best's disease, chronic retinal detachment, diseases associated with rubeosis, Eales' disease, proliferative vitreoretinopathy, familial exudative vitreoretinopathy, Stargardt's disease, presumed ocular histoplasmosis, hyperviscosity syndromes, myopia, post-laser complications, retinopathy of prematurity, infections causing a retinitis or choroiditis, optic pits, pars planitis, toxoplasmosis, choroidal neovascularization, Type 1 choroidal neovascularization, Type 2 choroidal neovascularization, Type 3 choroidal neovascularization, macular edema, cystoid macular edema, diabetic macular edema, ocular edema, glaucoma, neovascular glaucoma, surgery-induced edema, surgery-induced neovascularization, retinoschisis, retinal capillary occlusions, retinal angiomatous proliferation, vitreous hemorrhage, retinal neovascularization, polypoidal choroidal vasculopathy, juxtafoveal polypoidal choroidal vasculopathy, subfovial polypoidal choroidal vasculopathy, vitreomacular adhesion, geographic atrophy, retinal hypoxia, pathological myopia, dysregulated para-inflammation, chronic inflammation, chronic wound healing environment in the aging eye, carotid vacernous fistula, idiopathic occlusive arteriolitis, birdshot retinochoroidopathy, retinal vasculitis, incontinentia pigmenti, retinitis pigmentosa, tachyphylaxis, and limbal stem cell deficiency.

In certain such embodiments, the disease or condition of the eye is selected from retinopathy, ocular edema or ocular neovascularization. Thus, in certain embodiments, the invention relates to a method for treating or preventing a disease or condition of the eye comprising administering to a patient having a disease or condition of the eye a compound as disclosed herein, such as Compound 7, wherein the disease or condition of the eye is selected from retinopathy, ocular edema and ocular neovascularization.

In certain embodiments, the invention relates to a method for treating or preventing a disease or condition of the eye, comprising administering to a patient having a disease or condition of the eye a compound as disclosed herein, such as Compound 7, wherein the disease of condition can be a condition selected from the group consisting of diabetic macular edema, wet age-related macular degeneration, dry age-related macular degeneration, choroidal neovascularization, diabetic retinopathy, ocular ischemia, uveitis, retinal vein occlusion, central retinal vein occlusion, branch retinal vein occlusion, ocular trauma, surgery induced edema, surgery induced neovascularization, cystoid macular edema, ocular ischemia, and uveitis, comprising administering to a patient having a disease or condition of the eye selected from diabetic macular edema, wet age-related macular degeneration, dry age-related macular degeneration, choroidal neovascularization, diabetic retinopathy, ocular ischemia, uveitis, retinal vein occlusion, central retinal vein occlusion, branch retinal vein occlusion, ocular trauma, surgery induced edema, surgery induced neovascularization, cystoid macular edema, ocular ischemia, and uveitis.

In certain embodiments, the invention relates to a method for treating or preventing age-related macular degeneration, comprising administering to a patient having age-related macular degeneration a compound as disclosed herein, such as Compound 7. In certain such embodiments, the age-related macular degeneration is selected from wet age-related macular degeneration and dry age-related macular degeneration. In certain such embodiments, a patient with age-related macular degeneration may be at least about 50, at least about 55, at least about 60, at least about 65, or at least about 70 years old.

In certain embodiments, the invention relates to methods for treating or preventing a disease or condition characterized by unstable ocular vasculature of a diabetic patient, comprising administering to a diabetic patient having unstable ocular vasculature a compound as disclosed herein, such as Compound 7.

In certain embodiments, the invention relates to a method for treating or preventing diabetic macular edema, comprising administering to a patient having diabetic macular edema a compound as disclosed herein, such as Compound 7.

In certain embodiments, the invention relates to a method for treating or preventing diabetic retinopathy, comprising administering to a patient having diabetic retinopathy a compound as disclosed herein, such as Compound 7. In certain such embodiments, the diabetic retinopathy is proliferative. In certain embodiments, the diabetic retinopathy is non-proliferative.

In certain embodiments, the invention relates to a method for treating or preventing retinal vein occlusion, comprising administering to a patient having retinal vein occlusion a compound as disclosed herein, such as Compound 7.

In certain embodiments, the invention relates to a method for treating or preventing a disease or condition of the eye, comprising administering to a patient having a disease or condition of the eye, or a patient at risk of developing a disease or condition of the eye a pharmaceutically effective amount of a compound having a structure of Formula (I), Formula (II), Formula (III), Formula (IV), or of Formula (V), or a compound selected from Compound 1, Compound 2, Compound 3, Compound 4, Compound 5, Compound 6, Compound 7, Compound 8, Compound 9, Compound 10, Compound 11, Compound 12, and Compound 13 or a metabolite, e.g. Metabolite 1 or Metabolite 2, pharmaceutically acceptable salt, solvate, or hydrate thereof, wherein the compound or metabolite is administered topically, or systemically, or via injection into any portion of the eye, including subconjunctival, intravitreal, retrobulbar, intracameral, and subtenon injection, or using any other method or route of administration described herein, including ocular drug delivery systems, such as, but not limited to, colloidal dosage forms, such as nanoparticles, nanomicelles, liposomes, microemulsions, bioadhesive gels and fibrin sealant-based approaches, drug-eluting contact lenses, ultrasound-mediated drug delivery, ocular iontophoresis, and drug-coated microneedles.

In certain embodiments, provided herein are methods for treating or preventing choroidal neovascularization (CNV) in a subject comprising administering to said subject a therapeutically effective amount of Compound 7. In more specific embodiments, provided herein are methods for treating CNV in a subject comprising administering to said subject an effective amount of Compound 7, wherein the Compound 7 is administered by intravitreal injection. In certain embodiments, provided herein are methods for treating CNV in a subject comprising administering to said subject an effective amount of Compound 7, wherein the Compound 7 is administered in liquid form at a concentrations of about 0.01 mg/mL to about 0.1 mg/mL, or about 0.05 mg/mL to about 0.5 mg/mL, or about 0.1 mg/mL to about 1.0 mg/mL, or about 0.5 mg/mL to about 5 mg/mL, or about 1.0 mg/mL to about 10 mg/mL, or about 2 mg/mL to about 10 mg/mL, or about 5.0 mg/mL to about 10 mg/mL, or about 5.0 mg/mL to about 15 mg/mL, or about 10 mg/mL to about 20 mg/mL. In certain specific embodiments, provided herein are methods for treating CNV in a subject comprising administering to said subject an effective amount of Compound 7, wherein the Compound 7 is administered in liquid form at a concentration of 3.7 mg/mL or at concentration of 7.8 mg/mL. In certain specific embodiments, provided herein are methods for treating CNV in a subject comprising administering to said subject an effective amount of Compound 7, wherein the Compound 7 is administered in liquid form by intravitreal injection at a concentration of 3.7 mg/mL or at concentration of 7.8 mg/mL. In certain specific embodiments, provided herein are methods for treating CNV in a subject comprising administering to said subject an effective amount of Compound 7, wherein a single dose of Compound 7 is administered in liquid form by intravitreal injection at a concentration of 3.7 mg/mL or at concentration of 7.8 mg/mL. In certain specific embodiments, Compound 7 is administered topically.

All compounds described herein are contemplated to be used in the methods described herein and especially in the prevention or treatment of eye disorders and associated diseases.

### 5.4 Combination Therapy

In certain embodiments, a compound as disclosed herein, such as Compound 7 may be administered in combination with another medicament. Such combination therapy may be achieved by way of the simultaneous, sequential, or separate dosing of the individual components of the treatment. Additionally, when administered as a component of such combination therapy, a compound as disclosed herein, such as Compound 7 and the other medicament may be synergistic, such that the daily dose of either or both of the components may be reduced as compared to the dose of either component that would normally be given as a monotherapy. Alternatively, when administered as a component of such combination therapy, the compound as disclosed herein, such as Compound 7 and the other medicament may be additive, such that the daily dose of each of the components is similar or the same as the dose of either component that would normally be given as a monotherapy.

In certain embodiments, the other medicament is selected from a prostaglandin analog, beta-adrenergic receptor antagonist, alpha-2-adrenergic agonist, carbonic anhydrase inhibitor, miotic agent, monoclonal antibody, corticosteroid, glucocorticoid, kinase inhibitor, cycloplegic and an antimetabolite, or a combination thereof.

In certain embodiments, the other medicament is an anti-angiogenic medicament. In certain embodiments, the anti-angiogenic medicament selected from bevacizumab, aflibercept, ranibizumab, or pegaptanib sodium.

In certain embodiments, the other medicament is laser therapy.

In certain embodiments, the other medicament is an anti-platelet-derived growth factor (anti-PDGF) agent. In certain embodiments, the anti-platelet-derived growth factor agent is pegpleranib sodium. In certain embodiments, the anti-platelet-derived growth factor agent is Fovista®.

In certain embodiments, the other medicament is an anti-vascular endothelial growth factor (anti-VEGF) agent. In certain embodiments, the anti-vascular endothelial growth factor agent is Lucentis®, Avastin® Eylea® or Macugen®.

### 5.5 Patient Populations

In certain embodiments, the invention relates to treating a disease or condition of the eye, comprising administering to a patient having a disease or condition of the eye an effective amount of a compound disclosed herein, such as Compound 7, wherein, the patient is at least 50 years old, at least 60 years old, at least 65 years old, at least 70 years old, or even at least 80 years old. In certain embodiments, the patient is a geriatric patient. In certain embodiments, the patient is less than 18 years old. In certain embodiments, the patient is a pediatric patient. In certain embodiment, the patient is at least 18 years old.

In certain embodiments, the invention relates to treating a disease or condition of the eye, comprising administering to a patient having a disease or condition of the eye an effective amount of a compound disclosed herein, such as Compound 7, wherein, the patient is a member of a subpopulation selected from White, Hispanic, Black, and Asian.

In certain embodiments, the invention relates to treating a disease or condition of the eye, comprising administering to a patient having a disease or condition of the eye an effective amount of a compound disclosed herein, such as Compound 7, wherein, the patient has another disease or condition such as diabetes.

### 5.6 Doses and Dosing Regimens

In certain embodiments, a disease or condition as described herein, such as a disease or condition of the eye may be treated by administering to a patient having a disease or condition as described herein from about 0.01 mg/kg to about 500 mg/kg, about 0.01 mg/kg to about 50 mg/kg, about 0.1 mg/kg to about 10 mg/kg or about 0.1 mg/kg to about 5.0 mg/kg of a compound as disclosed herein, such as Compound 7. In certain such embodiments, the compound as described herein, such as Compound 7 is administered topically.

In certain embodiments, a disease or condition as described herein, such as a disease or condition of the eye may be treated by administering to a patient having a disease or condition as described herein from about 0.01 mg to about 500 mg, about 0.01 mg to about 50 mg, about 0.1 mg to about 10 mg or about 0.1 to about 5.0 mg of a compound as disclosed herein, such as Compound 7. In certain such embodiments, the compound as described herein, such as Compound 7 is administered topically.

In certain embodiments, a disease or condition as described herein, such as a disease or condition of the eye may be treated by administering to a patient having a disease or condition as described herein a daily dose of about 0.01 mg to about 500 mg, about 0.01 mg to about 50 mg, about 0.1 mg to about 10 mg, or about 0.1 to about 5 mg of a compound as disclosed herein, such as Compound 7. In certain such embodiments, the compound as described herein, such as Compound 7 is administered topically.

The suitability of compound provided herein, such as a compound having a structure of Formula (I), Formula (II), Formula (III), Formula (IV), or of Formula (V), or a compound selected from Compound 1, Compound 2, Compound 3, Compound 4, Compound 5, Compound 6, Compound 7, Compound 8, Compound 9, Compound 10, Compound 11, Compound 12, and Compound 13 or a metabolite, e.g. Metabolite 1 or Metabolite 2, pharmaceutically acceptable salt, solvate, or hydrate thereof for the treatment or prevention of a disease or condition of the eye, wherein the condition or disease can be a condition selected from the group consisting of retinopathy, diabetic retinopathy, radiation retinopathy, macular degeneration, age-related macular degeneration, early stage age-related macular degeneration, intermediate stage age-related macular degeneration, advanced stage age-related macular degeneration, Wet (exudative) age-related macular degeneration, specific genotypes associated with macular degeneration, cancer, solid tumors, blood borne tumors, choroidal melanoma, sickle cell retinopathy, neovascularization, ocular neovascularization, subretinal neovascularization, vein occlusion, retinopathy of prematurity, chronic uveitis/vitritis, ocular trauma, ocular ischemia, retinal ischemia, Best's disease, chronic retinal detachment, diseases associated with rubeosis, Eales' disease, proliferative vitreoretinopathy, familial exudative vitreoretinopathy, Stargardt's disease, presumed ocular histoplasmosis, hyperviscosity syndromes, myopia, post-laser complications, retinopathy of prematurity, infections causing a retinitis or choroiditis, optic pits, pars planitis, toxoplasmosis, choroidal neovascularization, Type 1 choroidal neovascularization, Type 2 choroidal neovascularization, Type 3 choroidal neovascularization, macular edema, cystoid macular edema, diabetic macular edema, ocular edema, glaucoma, neovascular glaucoma, surgery-induced edema, surgery-induced neovascularization, retinoschisis, retinal capillary occlusions, retinal angiomatous proliferation, vitreous hemorrhage, retinal neovascularization, polypoidal choroidal vasculopathy, juxtafoveal polypoidal choroidal vasculopathy, subfovial polypoidal choroidal vasculopathy, vitreomacular adhesion, geographic atrophy, retinal hypoxia, pathological myopia, dysregulated para-inflammation, chronic inflammation, chronic wound healing environment in the aging eye, carotid vacernous fistula, idiopathic occlusive arteriolitis, birdshot retinochoroidopathy, retinal vasculitis, incontinentia pigmenti, retinitis pigmentosa, tachyphylaxis, and limbal stem cell deficiency, can be confirmed by using the assays described in Section 5.8 below.

### 5.7 Pharmaceutical Compositions

Pharmaceutical compositions may be used in the preparation of individual, single unit dosage forms. Pharmaceutical compositions and dosage forms provided herein comprise a compound as provided herein, such as Compound 7. Pharmaceutical compositions and dosage forms can further comprise one or more excipients. Like the amounts and types of excipients, the amounts and specific types of active ingredients in a dosage form may differ depending on factors including, but not limited to, the route by which it is to be administered to patients.

In certain embodiments, administration of a compound as disclosed herein, such as Compound 7 may be by topical, oral or parenteral route. As used herein, the term “parenteral” includes intravitreous, intraocular, intracorneal, subcutaneous, intradermal, intravascular injections, such as intravenous, intramuscular and any another similar injection or infusion technique. In certain embodiments, a compound as disclosed herein, such as Compound 7 may be administered using an insertable or implantable device that is placed in the eye. In certain embodiments, a compound as disclosed herein, such as Compound 7 may be administered via a subconjunctival, subtenon, intracameral, retrobulbar, posterior juxtascleral route. In certain embodiments, a compound as disclosed herein, such as Compound 7 may be administered orally, such as in a tablet or capsule formulation. In certain embodiments, a compound as disclosed herein may be administered topically, such as a topical ophthalmic solution (eye drop).

### 5.7.1 Topical Ocular Formulations

Disclosed herein are formulations comprising the disclosed compounds as topical ophthalmic solutions (eye drops), which are normally available as a sterile, isotonic (i.e., a pH of between about 3 and about 8, between about 4 to about 8, between about 7 to about 8, or about 7.4) solution, optionally further comprising a preservative.

The term “eye drops” as used herein refers to a pharmaceutical liquid formulation which is administered in the form of drops on the external surface of the eye and which has a local effect on the posterior segment of the eye, including the choroids, retinal pigment epithelium, retina, macula, fovea, optic nerve and vitreous humor.

Accordingly, in certain embodiments, a compound as disclosed herein, such as Compound 7 may be combined with purified water and adjusted for physiological pH and isotonicity. Examples of buffering agents to maintain or adjust pH include, but are not limited to, acetate buffers, citrate buffers, phosphate buffers and borate buffers. Examples of tonicity adjustors are sodium chloride, mannitol and glycerin.

The eye drop formulation is then optionally aliquoted into either a plurality of discrete, sterile disposable cartridges each of which is suitable for unit dosing, or a single cartridge for unit dosing. Such a single disposable cartridge may be, for example, a conical or cylindrical specific volume dispenser, with a container having side-walls squeezable in a radial direction to a longitudinal axis in order to dispense the container contents therefrom at one end of the container. Such disposable containers are currently used to dispense eye drops at 0.3 to 0.4 mL per unit dosing, and are ideally adaptable for the delivery of eye drops.

Ophthalmic eye-drop solutions may also be packaged in multi-dose form, for example, as a plastic bottle with an eye-dropper. In such formulations, preservatives are optionally added to prevent microbial contamination after opening of the container. Suitable preservatives include, but are not limited to: benzalkonium chloride, thimerosal, chlorobutanol, methylparaben, propylparaben, phenylethyl alcohol, edetate disodium, sorbic acid, polyquatemium-1, or other agents known to those skilled in the art, and all of which are contemplated for use in the present invention. Preservative-containing formulations may comprise from about 0.001 to about 1.0% weight/volume of the preservative.

In certain embodiments, polymers may be added to ophthalmic solutions in order to increase the viscosity of the vehicle, thereby prolonging contact of the solution with the cornea and enhancing bioavailability. In certain embodiments, such polymers are selected from cellulose derivatives (e.g., methylcellulose, hydroxyethylcellulose, hydroxypropylcellulose or carboxymethylcellulose), dextran 70, gelatin, polyols, glycerin, polyethylene glycol 300, polyethylene glycol 400, polysorbate 80, propylene glyclol, polyvinyl alcohol and povidone, or a combination thereof

In certain embodiments ophthalmic solutions as disclosed herein may further comprise stabilizer/solubilizer such as a cyclodextrin. In certain such embodiments, the cyclodextrin is selected from α-cyclodextrin, β-cyclodextrin, γ-cyclodextrin, hydroxypropyl-β-cyclodextrin, hydroxypropyl-γ-cyclodextrin, dimethyl-β-cyclodextrin and dimethyl-γ-cyclodextrin.

In certain embodiments, a compound as disclosed herein, such as Compound 7 may be administered in a sustained release ophthalmic solution formulation.

In certain embodiments, the compound as disclosed herein may be administered through ocular drug delivery systems, such as, but not limited to, colloidal dosage forms, such as nanoparticles, nanomicelles, liposomes, microemulsions, bioadhesive gels and fibrin sealant-based approaches to sustain drug levels at the target site. Other ocular drug delivery systems include drug-eluting contact lenses, ultrasound-mediated drug delivery, ocular iontophoresis, and drug-coated microneedles.

In certain embodiments, the frequency of administration can vary greatly, depending on the needs of each subject and the severity of the disease to be treated, such administration may be from about once a week to about ten times a day, such as from about three times a week to about three times a day, or once or twice a day.

### 5.7.2 Oral Formulations

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM. Other suitable forms of microcrystalline cellulose include, but are not limited to, silicified microcrystalline cellulose, such as the materials sold as PROSOLV 50, PROSOLV 90, PROSOLV HD90, PROSOLV 90 LM, and mixtures thereof

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

In certain embodiments, fillers may include, but are not limited to block copolymers of ethylene oxide and propylene oxide. Such block copolymers may be sold as POLOXAMER or PLURONIC, and include, but are not limited to POLOXAMER 188 NF, POLOXAMER 237 NF, POLOXAMER 338 NF, POLOXAMER 437 NF, and mixtures thereof.

In certain embodiments, fillers may include, but are not limited to isomalt, lactose, lactitol, mannitol, sorbitol xylitol, erythritol, and mixtures thereof.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, povidone, crospovidone, polacrilin potassium, sodium starch glycolate, potato or tapioca starch, other starches, pre-gelatinized starch, other starches, clays, other algins, other celluloses, gums, and mixtures thereof.

Glidants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium stearyl fumarate, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional glidants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic colloidal silicon dioxide product sold by Cabot Co. of Boston, Mass.), and mixtures thereof. If used at all, glidants may be used in an amount of less than about 1 weight percent of the pharmaceutical compositions or dosage forms into which they are incorporated.

In certain embodiments, an oral dosage form comprises the compound, silicified microcrystalline cellulose, sodium starch glycolate, a block copolymer of ethylene oxide and propylene oxide, sodium stearyl fumarate and colloidal silicon dioxide. In certain embodiments, an oral dosage form comprises the Compound (I) in an amount of about 5% to about 75% by weight, silicified microcrystalline cellulose in an amount of about 15% to about 85%, sodium starch glycolate in an amount of about 2% to about 10%, block copolymer of ethylene oxide and propylene oxide in an amount of about 2% to about 10%, sodium stearyl fumarate in an amount of 0.2% to about 2%, and colloidal silicon dioxide in an amount of about 0.2% to about 2% by weight of the oral dosage form.

In certain embodiments, an oral dosage form comprises the compound, microcrystalline cellulose, isomalt, sodium starch glycolate, sodium lauryl sulfate, povidone, colloidal silicon dioxide, and magnesium stearate. In certain embodiments, an oral dosage form comprises the Compound 7 in an amount of about 40% to about 50%, microcrystalline cellulose in an amount of about 40% to about 50%, isomalt in an amount of 0% to about 5%, sodium starch glycolate in an amount of about 5% to about 10%, sodium lauryl sulfate in an amount of 0.2% to about 2%, povidone in an amount of about 2% to about 10%, colloidal silicon dioxide in an amount of 0.1% to about 1%, and magnesium stearate in an amount of about 0.1% to about 1% by weight of the oral dosage form.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof

### 5.8 Examples

### 5.8.1 CNV Model—Intravitreal Injection of Low and High Dose Salt in Solution with Vehicle Control

The efficacy of the compounds, such as Compound 7, in treating choroidal neovascularization (CNV) is demonstrated in a rodent model of surgically induced CNV. In one example, twenty-four (24) naïve, male Brown Norway rats, 225-250 g at study start, are acclimated for a minimum of 5 days prior to dosing. Animals are weighed prior to dosing and at euthanasia. All animals undergo pre-screening ophthalmic examinations prior to study start as follows:

A. Slit-lamp biomicroscopy and indirect ophthalmoscopy

B. Ocular findings analysis using the McDonald-Shadduck scoring system

C. Inclusion criterion: Only animals with a score of “0” included in study

CNV is then induced via laser treatment (e.g. laser photocoagulation (20-100-μm spot size; 0.05-0.1 seconds duration; 50 to 200 mW) at six positions of the posterior pole in each eye) by a board certified veterinary ophthalmologist. Only animals in which the laser produces a bubble indicating rupture of Bruch's membrane, are included in the study. The compound is administered in suitable form of a Test Article. Test and Control Article treatment begin 3 days post laser induction. In another experiment, the Test and Control Article treatments begin prior to or immediately post induction, in yet other experiments, Test and Control Article treatments begin 10 days post induction. Study duration is 3 weeks, with the option to extend on a weekly basis based on extent of healing. Animals have clinical ophthalmic examinations and fluorescein angiography approximately 3 days post-induction (prior to Test Article treatment) to confirm disease state. The route of administration for Test Article(s) and vehicle control is intravitreal (IVT) injection on Day 1, which is approximately 3-4 days post-induction, once the CNV disease state has been confirmed.

In the first experiment, a low dose (5 μL at 3.7 mg/mL) and a high dose (5 μL at 7.8 mg/mL) of the compound are evaluated against vehicle control. The three treatment groups are (N=8 per group, 24 animals total):

A. Test Article—Low Dose (19 μg/eye)

B. Test Article—High Dose (39 μg/eye)

C. Vehicle Control

Daily gross ocular observations and general health observations are performed. Additional exams, performed weekly up to 3 weeks post Test Article dosing, include clinical ophthalmic exams, slit-lamp biomicroscopy, indirect ophthalmoscopy, and fluorescein staining if needed. Ocular findings are scored using the McDonald-Shadduck scoring system.

Fluorescein angiography (e.g. using Heidelberg SPECTRALIS® instrumentation) is performed to determine the degree of neovascularization (NV) in the retina and/or choroid. After fluorescein angiography, tissues are then harvested for possible future analysis (e.g. eyes are fixed in Davidson's solution and then transferred to 70% ethanol). Alternatively, the area of retinal and/or choroidal NV can be determined in various other ways. In one example, prior to sacrifice, the rodents are perfused with PBS containing 50 mg/mL of fluorescein-labeled dextran (2×10⁶ Da average molecular mass). Alternatively, fluorescein-labeled Lycopersicon esculentum (tomato) lectin can be used. The choroids are then flat mounted and examined by fluorescence microscopy. A standard image analysis software is then used to measure the total area of retinal/choroidal NV with the investigator masked with respect to treatment group.

Alternatively, for post sacrifice analysis, retinas/choroids are dissected intact and washed with PBS. After blocking with animal serum, retinas are stained with FITC-labeled Griffonia simplicifolia (GSA) lectin. Retinas/choroids are then flat mounted and digital photographs obtained. Images are edited to show the entire retina. A standard image analysis software can be used to measure the area of retinal/choroidal NV per retina/choroid by an investigator blinded with respect to treatment group. Other optional procedures include toluidine blue staining, mouse platelet endothelial cell adhesion molecule-1 (PECAM-1) antibody staining, and selective staining of retinal NV and hyaloid vessels for light microscopy. Areas of NV can be calculated and plotted against serum levels of the compound.

Intravascular lumens can be visualized using peroxidase perfusion in the living animal with subsequent histologic analysis. For example, animals are anesthetized and 50 mg horseradish peroxidase 200 μL PBS are injected into the jugular vein. Animals are killed and the eyes enucleated and fixed. The anterior part of the eye, vitreous, and retina are removed, and the posterior eye cup fixed and embedded. Thin sections are stained with uranyl acetate and lead citrate, and then examined by electron microscopy. In addition, alkaline phosphatase can be visualized in endothelial cells. Eyes are enucleated and the posterior half of the eye kept while retina and retinal pigment epithelium are removed. The tissue is washed, and after fixation, the tissues are washed in 0.1 M cacodylate. Tissues are incubated with a solution consisting of 40 mL 0.1 M Tris buffer with 20 mg fast blue RR salt and 4 mg naphthol AS-MX phosphate dissolved in 0.2 mL dimethyl sulfoxide. The tissues are washed and postfixed, bleached and washed again. Tissues are then flat mounted on slides for light microscopy.

A number of alternative primary antibodies can be used for the immunohistochemical analysis of NV: biotinylated isolectin B4 (binds galactosyl epitopes on the membranes of i.a. endothelial cells); rat anti-CD31 (adhesion molecule expressed by i.a. vascular endothelial cells); rabbit anti-von Willebrand factor (protein expressed by endothelial cells and platelets); rat anti-CD105 (a regulatory component of the TGF-β receptor complex expressed by endothelial cells); rat anti-ICAM-2 (an intercellular adhesion molecule mainly found on resting endothelial cells); rabbit anti-desmin; and rat anti-MECA32 (an antigen specific for endothelial cells). Furthermore, NV may be evaluated by staining the pericytes with rabbit anti-NG2 (a chondroitin sulfate proteoglycan expressed on the surfaces of vascular mural cells during normal and pathologic angiogenesis). In addition, the vascular basement membrane can be stained with rabbit anti-collagen IV (one of the several protein families included in the matrix components of vascular basement membrane).

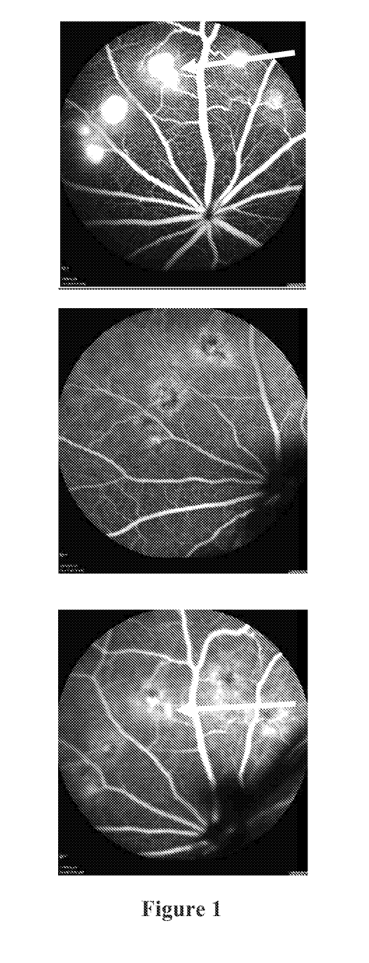

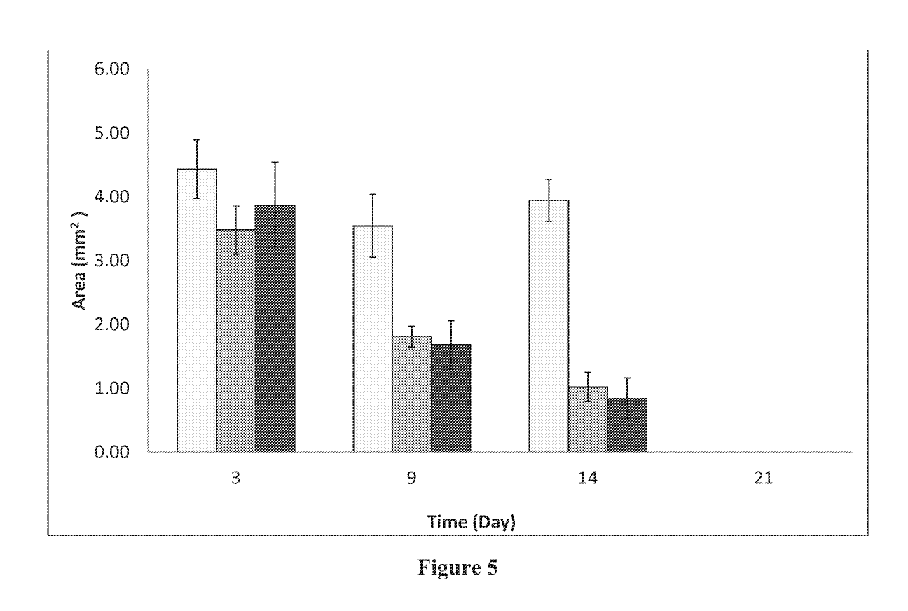

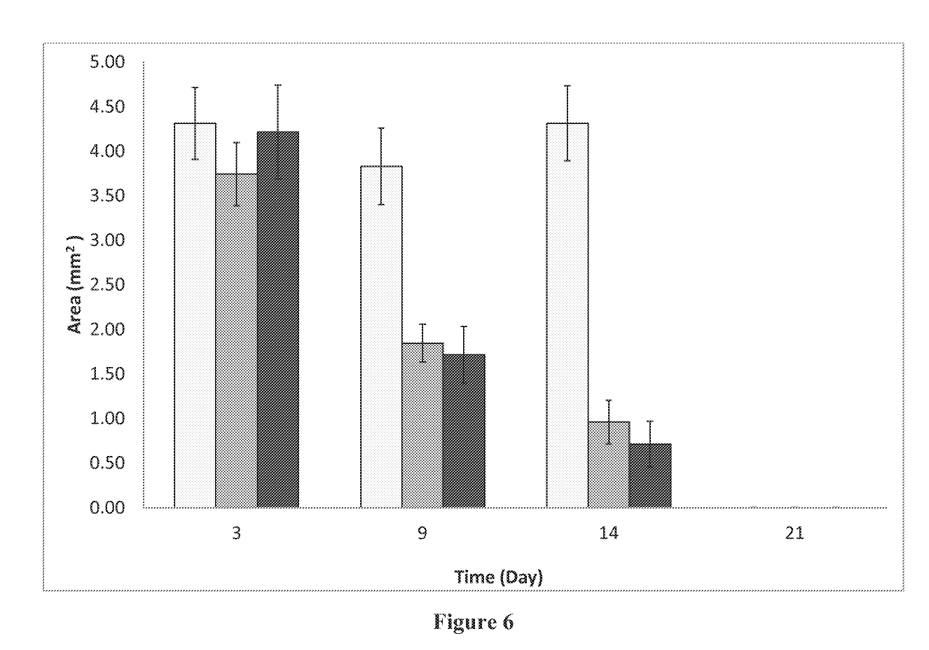

Results from an initial experiment treating animals with a control vehicle, low dose (19 μg/eye), or high dose (39 μg/eye) of Compound 7 are shown in FIGS. 1-6.

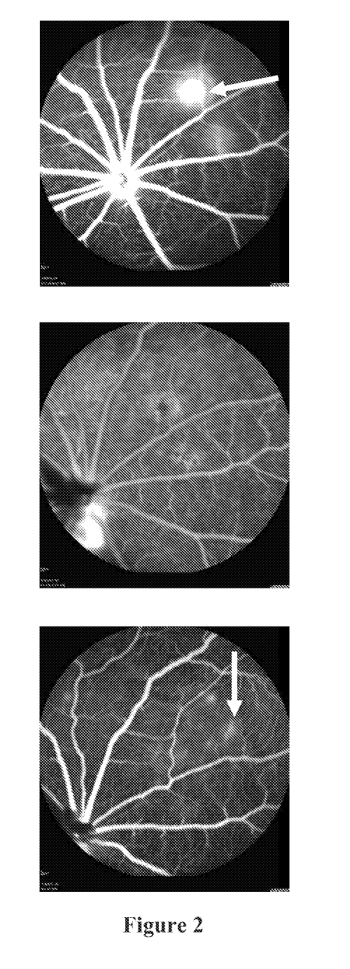

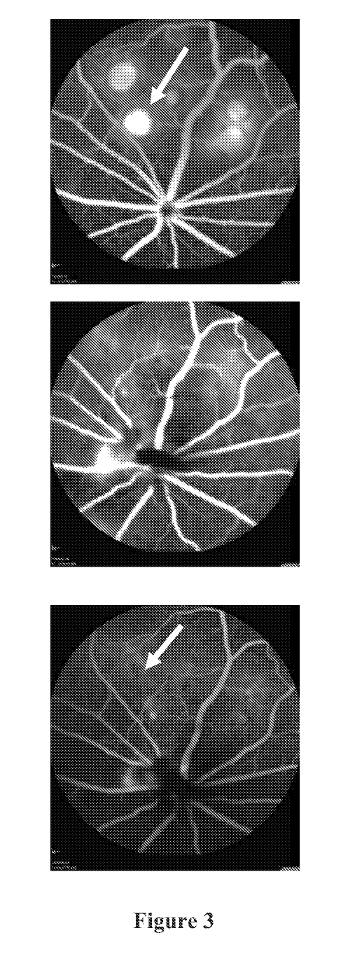

Animals were dosed on Day 3. FIG. 1 shows the results for a sample animal of the vehicle control group. The surrounding regions around the laser burn continued to show a higher fluorescence from Days 3-14. Day 9 intensities were attenuated due to the presence of fluorescein in both the anterior and posterior chambers of the eye. FIG. 2 shows the results for a sample animal of the Low Dose group. This animal exhibited fluorescein leakage that started out high during the initial onset pre-dose on Day 3 (top), attenuated over time, and almost subsided completely by Day 14 (bottom). FIG. 3 shows the results for a sample animal of the High Dose group. This animal exhibited high fluorescein leakage during the initial onset pre-dose on Day 3 (top), but that no fluorescein leakage was observed on Day 14 (bottom). Note that an exogenous retinal detachment from the initial laser burn was observed in the left eye (OS).

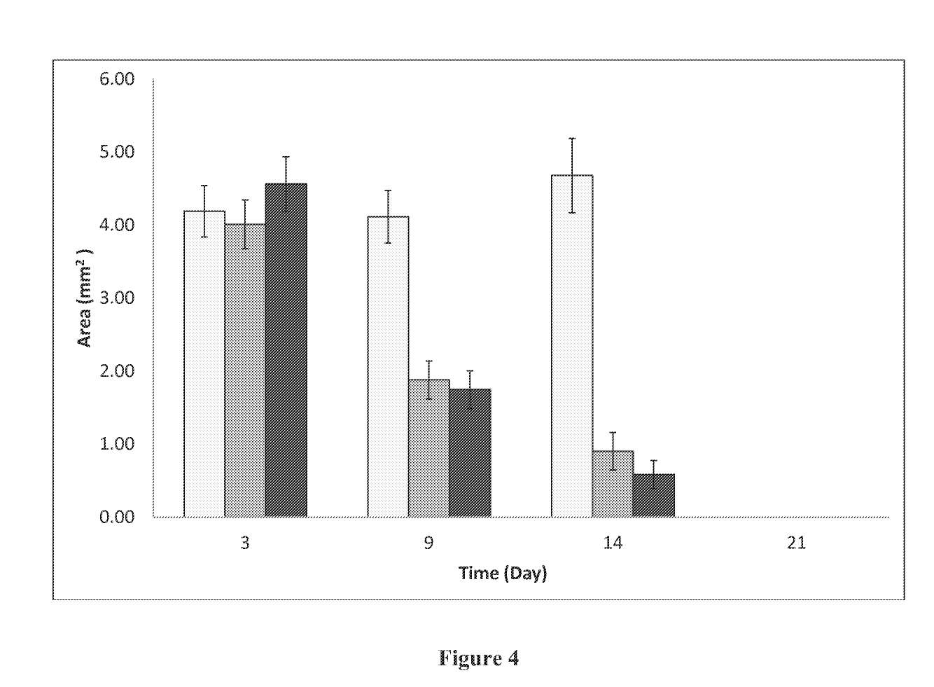

FIGS. 4-6 and Tables 1-3 below show Mean (±SEM) of lesion areas in the right, left and both eyes, respectively. It appears that animals treated with low or high doses of Compound 7 had reduced overall fluorescence and leakage when compared to animals treated with vehicle control.

TABLE 1

| | Right Eye | | | | | |
|---|---|---|---|---|---|---|
| | Mean | | | SEM | | |
| | Day 3 | Day 9 | Day 14 | Day 3 | Day 9 | Day 14 |
| Vehicle | 4.19 | 4.11 | 4.68 | 0.35 | 0.36 | 0.51 |
| Low Dose | 4.01 | 1.88 | 0.90 | 0.33 | 0.26 | 0.26 |
| High Dose | 4.56 | 1.75 | 0.58 | 0.37 | 0.26 | 0.19 |

TABLE 2

| | Left Eye | | | | | |
|---|---|---|---|---|---|---|
| | Mean | | | SEM | | |
| | Day 3 | Day 9 | Day 14 | Day 3 | Day 9 | Day 14 |
| Vehicle | 4.43 | 3.55 | 3.94 | 0.45 | 0.49 | 0.33 |
| Low Dose | 3.48 | 1.81 | 1.02 | 0.37 | 0.16 | 0.23 |
| High Dose | 3.86 | 1.68 | 0.84 | 0.68 | 0.38 | 0.33 |

TABLE 3

| | Both Eyes | | | | | |
|---|---|---|---|---|---|---|
| | Mean | | | SEM | | |
| | Day 3 | Day 9 | Day 14 | Day 3 | Day 9 | Day 14 |
| Vehicle | 4.31 | 3.83 | 4.31 | 0.40 | 0.43 | 0.42 |
| Low Dose | 3.74 | 1.84 | 0.96 | 0.35 | 0.21 | 0.24 |
| High Dose | 4.21 | 1.71 | 0.71 | 0.53 | 0.32 | 0.26 |

Table 4 shows the percentage area reduction of lesion areas in the right, left and both eyes, respectively. It appears that animals treated with low or high doses of Compound 7 had reduced overall fluorescence and leakage when compared to animals treated with vehicle control.

TABLE 4

| | Right Eye | | | Left Eye | | | Both Eyes | | |
|---|---|---|---|---|---|---|---|---|---|
| | Day 3 | Day 9 | Day 14 | Day 3 | Day 9 | Day 14 | Day 3 | Day 9 | Day 14 |
| Vehicle | 0.00 | 7.64 | 21.48 | 0.00 | −18.07 | −5.17 | 0.00 | −5.21 | 8.15 |
| Low | 0.00 | −52.47 | −79.25 | 0.00 | −44.39 | −68.00 | 0.00 | −48.43 | −73.63 |
| High | 0.00 | −61.00 | −87.49 | 0.00 | −46.66 | −79.77 | 0.00 | −53.83 | −83.63 |

### 5.8.2 CNV Model—Intravitreal Injection of Low and High Dose Salt in Solution with and without Anti-VEGF Combination

In another experiment, the compound is evaluated at low and high dose in combination with an intravitreally administered anti-vascular endothelial growth factor (anti-VEGF) agent such as Lucentis®, Avastin®, Eylea® or Macugen®. For example, the Test Article is delivered in combination with 2.5 μL Eylea® in a single injection. The remaining experimental procedure is carried out as described in 5.8.1 above, but with modified treatment groups. A low dose (2.5 μL at 7.4 mg/mL) and a high dose (2.5 μL at 15.6 mg/mL) of the compound alone or in combination are evaluated against vehicle control. The treatment groups are (N=8 per group, 40 animals total):

A. Test Article—Low Dose (2.5 μL; 19 μg/eye)+2.5 μL Vehicle

B. Test Article—Low Dose (2.5 μL; 19 μg/eye)+2.5 μL Eylea®

C. Test Article—High Dose (2.5 μL; 39 μg/eye)+2.5 μL Vehicle

D. Test Article—High Dose (2.5 μL; 39 μg/eye)+2.5 μL Eylea®

E. Vehicle Control

The imaging and analyses are carried out as described in 5.8.1 above.

### 5.8.3 CNV Model—Topical Application at Various Dosing Regimens

Another experiment demonstrates the efficacy of the compound in treating CNV when the compound is delivered topically in a rodent model of surgically induced CNV. The compound is evaluated at low, medium and high dose, and compared with an intravitreally administered anti-vascular endothelial growth factor (anti-VEGF) agent such as Lucentis®, Avastin®, Eylea®, or Macugen®.

One experiment is carried out in 46 young female Dutch Belted rabbits. Ocular examinations are done by a board certified veterinary ophthalmologist by slitlamp and indirect ophthalmoscopy to exclude animals with anterior segment defects. The success rate is estimated to be approximately 70%.

CNV is induced in up to 42 animals and only 30 of the animals with well-defined CNV lesions at approximately week 3 are included in the study. CNV is induced with subretinal injection of heparin-sepharose beads with fibroblast growth factor and Lipopolysaccharide (100 ng bFGF, 100 ng LPS in 50 μL) in PBS, performed by a veterinary ophthalmologist. Animals are treated with NSAID and or buprenorphine for up to 3 weeks or until anterior chamber inflammation subsides.

Dose administration is initiated when CNV lesions are well defined and intraocular inflammation subsides (˜3 weeks). The VEGF inhibitor or antibody is given once intravitreously to the right eyes. Topical ocular doses are administered by beginning of week 4 with BID dosing, approximately 8 hours apart for four weeks (to day 56). In the first experiment, a low dose (50 μL at 3.7 mg/mL), a medium dose (50 μL at 5.7 mg/mL) and a high dose (50 μL at 7.8 mg/mL) of the compound are evaluated against vehicle control. The treatment groups are (N=6 per group, 30 animals total):

A. Test Article—Low Dose (190 μg/eye)

B. Test Article—Medium Dose (285 μg/eye)

C. Test Article—High Dose (390 μg/eye)

D. 50 μL Eylea®

E. Vehicle Control

Ophthalmic examinations (slitlamp and indirect) are performed pre-dose, and at days 1, 3, 8, 15, 22, 43, and 56. Ocular fluorescein angiography is performed on Weeks 2 (FITC Dextran), 3, 6, and 8. Animals in Groups D and E are maintained for possible further evaluation at later time points. Animals in Groups A, B, and C are sacrificed and blood (approximately 5 mL) is collected from each animal. Both eyes are collected from all animals following sacrifice. The globes are excised and choroid, retina and plasma are collected, and each sample is weighed. Image analysis of the angiograms (tracing of lesion areas and semi-quantitative grading of fluorescein leakage) is also performed. Analysis of flat mount samples is generally carried out as described above.

In another experiment, the experimental procedures are generally performed as above, but with the administration of the compound at two dose levels, and administered 1× and 2× daily. The treatment groups are (N=6 per group, 24 animals total):

A. Test Article—Low Dose (190 μg/eye)—1× per day

B. Test Article—Low Dose (190 μg/eye)—2× per day

C. Test Article—High Dose (390 μg/eye)—1× per day

D. Test Article—High Dose (390 μg/eye)—2× per day

The imaging and analyses are carried out as described above.

### 5.8.4 CNV Model—Topical Application at Two Dose Levels with and without Anti-VEGF Combination

Another rabbit experiment demonstrates the efficacy of the compound in treating CNV when delivered topically in a model of surgically induced CNV in combination with an anti-vascular endothelial growth factor (anti-VEGF) agent such as Lucentis®, Avastin®, Eylea®, or Macugen®. For example, the Test Article is delivered topically in combination with a single injection of ≦50 μL Eylea®. The compound is evaluated at low and high dose, alone and in combination with the anti-VEGF agent. The remaining experimental procedure is carried out as described in 5.8.3 above, but with modified treatment groups. In the first experiment, a low dose (50 μL at 3.7 mg/mL) and a high dose (50 μL at 7.8 mg/mL) of the compound co-administered with 25 μL Eylea® are evaluated against vehicle control. The treatment groups are (N=6 per group, 30 animals total):

A. Test Article—Low Dose (190 μg/eye)

B. Test Article—Low Dose (190 μg/eye)+25 μL Eylea®

C. Test Article—High Dose (390 μg/eye)

D. Test Article—High Dose (390 μg/eye)+25 μL Eylea®

E. Vehicle Control

The imaging and analyses are carried out as described in 5.8.3 above.

### 5.8.5 Hypoxia Model—Topical Application at Two Dose Levels

The efficacy of the compound in treating CNV is demonstrated in a rodent model of oxygen-induced ischemic retinopathy. In variation to the methods described above, ischemic retinopathy is produced by exposing mice to a period of hyperoxia. It has been shown previously that exposure of mice at postnatal day 7 (P7) to a continuous treatment of 75% oxygen for 5 days, followed by return to normal room air, resulted in reproducible and quantifiable retinal NV without hypertrophy or dilatation of the hyaloid vessels. Accordingly, in this experiment, mice at P7 are exposed to hyperoxia (75% oxygen). At P12, the mice are returned to room air and given daily topical doses as follows: a low dose (5 μL at 3.7 mg/mL) and a high dose (5 μL at 7.8 mg/mL) of the compound, or 5 μL vehicle control. The treatment groups are (N=8 per group, 24 animals total):

A. Test Article—Low Dose (19 μg/eye)

B. Test Article—High Dose (39 μg/eye)

C. Vehicle Control

At P17, the NV of the retina and/or choroid is measured and eyes are evaluated as described in 5.8.3 above. In an alternative analysis, P17 mice are given an intraocular injection of 1 μL of rat anti-mouse platelet endothelial cell adhesion molecule-1 (PECAM-1) antibody. Secondary and tertiary antibodies may be used, e.g. biotinylated goat anti-rat IgG as secondary antibody, and Cy3-labeled streptavidin as tertiary antibody. After 12 h, the mice are euthanized, and eyes are fixed in formalin. Retinas are dissected, washed, and incubated with goat-anti rat polyclonal antibody conjugated with Alexa 488 and flat mounted. A standard image analysis software can be used to measure the area of retinal/choroidal NV per retina/choroid by an investigator blinded with respect to treatment group.

### 5.8.6 RhoVEGF Model—Topical Application at Optimal Doses to Determine Whether the Effect is Fully Dependent on VEGF

It has been shown previously that transgenic mice in which the rhodopsin promoter drives expression of VEGF in photoreceptors (Rho-VEGF mice) produce NV that originates from retinal vessels and grows into the subretinal space through the photoreceptor layer. The development of sprouts of NV in neonatal transgenic mice starts at P10. In this experiment, at P15, hemizygous Rho-VEGF mice are given daily topical doses of 5 μL at 7.8 mg/mL of the Test Article, or saline control until P21. The remaining experimental procedure is carried out as described in 5.8.5 above, but with modified treatment groups. The treatment groups are (N=8 per group, 16 animals total):

A. Test Article—(39 μg/eye)

B. Vehicle Control

At P21, the NV of the retina and/or choroids in each group is measured and analyzed as described in 5.8.3 above. In alternative experiment, double transgenic Rho/rtTA-TRE/VEGF mice, wherein the Rho promoter is combined with the rtTA system to direct doxycyline-inducible expression of VEGF in photoreceptors, are used instead of Rho-VEGF mice, and examined as described in this sub-section 5.8.6.

While particular embodiments of the present disclosure have been illustrated and described, those skilled in the art could routinely make various changes and modifications without departing from the spirit and scope of the disclosure. It is therefore intended to cover in the appended claims all such changes and modifications that are within the scope of this disclosure. All references cited herein are incorporated herein by reference.

1. A method for treating a disease or condition of the eye, comprising administering to a patient having a disease or condition of the eye a compound having a structure of Formula (I): 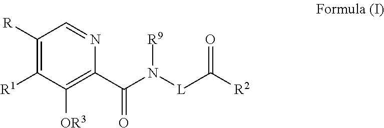
or a pharmaceutically acceptable salt, solvate or hydrate thereof, wherein
R and R¹ are each independently:
(i) hydrogen
(ii) substituted or unsubstituted phenyl; or
(iii) substituted or unsubstituted heteroaryl;
said substitution selected from:
(i) C₁-C₄ alkyl;
(ii) C₃-C₄ cycloalkyl;
(iii) C₁-C₄ alkoxy;
(iv) C₃-C₄ cycloalkoxy;
(v) C₁-C₄ haloalkyl;
(vi) C₃-C₄ halocycloalkyl;
(vii) halogen;
(viii) cyano;
(ix) NHC(O)R⁴;
(x) C(O)NR^{5a}R^{5b}; and
(xi) heteroaryl; or
(xii) two substituents are taken together to form a fused ring having from 5 to 7 atoms;
R⁴ is a C₁-C₄ alkyl or C₃-C₄ cycloalkyl;
R^{5a} and R^{5b} are each independently selected from:
(i) hydrogen;
(ii) C₁-C₄ alkyl;
(iii) C₃-C₄ cycloalkyl; or
(iv) R^{5a} and R^{5b} are taken together to form a ring having from 3 to 7 atoms;
R² is selected from:
(i) OR⁶
(ii) NR^{7a}R^{7b}; and
R⁶ is selected from hydrogen and C₁-C₄ alkyl or C₃-C₄ cycloalkyl;
R^{7a} and R^{7b} are each independently selected from:
(i) hydrogen;
(ii) C₁-C₄ alkyl or C₃-C₄ cycloalkyl; or
(iii) R^{7a} and R^{7b} are taken together to form a ring having from 3 to 7 atoms;
R³ is selected from hydrogen, methyl, and ethyl;
L is a linking unit having a structure —[C(R^{8a}R^{8b})]ₙ—
R^{8a} and R^{8b} are each independently selected from hydrogen, methyl and ethyl;
n is an integer from 1 to 3; and
R⁹ is selected from hydrogen and methyl,
wherein the disease or condition of the eye is a condition selected from the group consisting of radiation retinopathy, age-related macular degeneration early stage age-related macular degeneration, intermediate stage age-related macular degeneration, advanced stage age-related macular degeneration, Wet (exudative) age-related macular degeneration, specific genotypes associated with macular degeneration, cancer, choroidal melanoma, sickle cell retinopathy, subretinal neovascularization, choroidal neovascularization, Type 1 choroidal neovascularization, Type 2 choroidal neovascularization, Type 3 choroidal neovascularization, macular edema, cystoid macular edema, diabetic macular edema, ocular edema, glaucoma, neovascular glaucoma, surgery-induced edema, surgery-induced neovascularization, retinoschisis, retinal capillary occlusions, retinal angiomatous proliferation, vitreous hemorrhage, retinal neovascularization, polypoidal choroidal vasculopathy, juxtafoveal polypoidal choroidal vasculopathy, subfovial polypoidal choroidal vasculopathy, vitreomacular adhesion, geographic atrophy, retinal hypoxia, pathological myopia, dysregulated para-inflammation, chronic inflammation, chronic wound healing environment in the aging eye, carotid vacernous fistula, idiopathic occlusive arteriolitis, birdshot retinochoroidopathy, retinal vasculitis, incontinentia pigmenti, retinitis pigmentosa, tachyphylaxis, and limbal stem cell deficiency.

2. The method of claim 1, wherein the disease or condition of the eye is characterized by changes in the ocular vasculature.

3. The method of claim 1, wherein the disease or condition of the eye is selected from retinopathy, ocular edema and ocular neovascularization

4. The method of claim 1, wherein the disease or condition of the eye is selected from diabetic macular edema, age-related macular degeneration, choroidal neovascularization, diabetic retinopathy, ocular ischemia, uveitis, retinal vein occlusion, ocular trauma, surgery induced edema, surgery induced neovascularization, cystoid macular edema, ocular ischemia, and uveitis.

5. The method of claim 1, wherein the disease or condition of the eye is age-related macular degeneration.

6. The method of claim 5, wherein the age-related macular edema is selected from wet age-related macular degeneration and dry age-related macular degeneration.

7. The method of claim 5 or 6, wherein the patient having age-related macular degeneration is at least about 50 years old.

8. The method of claim 7, wherein the patient having age-related macular degeneration is at least about 55 years old.

9. The method of claim 8, wherein the patient having age-related macular degeneration is at least about 60 years old.

10. The method of claim 9, wherein the patient having age-related macular degeneration is at least about 65 years old.

11. The method of claim 10, wherein the patient having age-related macular degeneration is at least about 70 years old.

12. The method of claim 1, wherein the disease or condition of the eye is characterized by unstable ocular vasculature of a diabetic patient.

13. The method of claim 1, wherein the disease or condition of the eye is diabetic macular edema.

14. The method of claim 1, wherein the disease or condition of the eye is diabetic retinopathy.

15. The method of claim 14, wherein the diabetic retinopathy is proliferative.

16. The method of claim 14, wherein the diabetic retinopathy is non-proliferative.

17. The method of claim 1, wherein the disease or condition of the eye is retinal vein occlusion.

18. The method of any one of claims 1 to 17, wherein the compound has a structure: 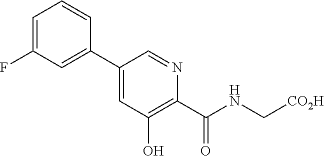 [SMILES: O=C(O)CNC(=O)c1ncc(-c2cccc(F)c2)cc1O]
or a pharmaceutically acceptable salt, solvate or hydrate thereof.

19. The method of any one of claims 1 to 18, wherein the compound is administered topically.

20. The method of any one of claims 1 to 19, wherein the compound of Formula (I) is administered in combination with another medicament.

21. The method of claim 20, wherein the other medicament is selected from a prostaglandin analog, beta-adrenergic receptor antagonist, alpha-2-adrenergic agonist, carbonic anhydrase inhibitor, miotic agent, monoclonal antibody, corticosteroid, glucocorticoid, kinase inhibitor, cycloplegic and antimetabolite, or a combination thereof.

22. The method of claim 20, wherein the other medicament is an anti-angiogenic medicament.

23. The method of claim 22, wherein the other medicament is an anti-angiogenic medicament selected from bevacizumab, aflibercept, ranibizumab, or pegaptanib sodium.

24. The method of claim 20, wherein the other medicament is laser therapy.

25. The method of claim 20, wherein the other medicament is an anti-platelet-derived growth factor.

26. The method of claim 25, wherein the anti-platelet-derived growth factor is Fovista®.

27. The method of claim 20, wherein the other medicament is an anti-vascular endothelial growth factor.

28. The method of claim 27, wherein the anti-vascular endothelial growth factor is Lucentis®, Avastin®, Eylea®, or Macugen®.
